(19) United States
(12) Patent Application Publication
(10) Pub. No.: US 2025/0361303 A1
(43) Pub. Date: Nov. 27, 2025

(54) FORMULATIONS FOR ANTI-PD-L1/ANTI-4-1BB BISPECIFIC ANTIBODIES

(71) Applicant: BEIJING HANMI PHARMACEUTICAL CO., LTD., Beijing (CN)

(72) Inventors: Lanxin ZHANG, Beijing (CN); Linlin TAN, Beijing (CN); Fei FAN, Beijing (CN); Haitao GU, Beijing (CN); Jiawang LIU, Beijing (CN); Kyoungwoo LEE, Beijing (CN)

(73) Assignee: BEIJING HANMI PHARMACEUTICAL CO., LTD., Beijing (CN)

(21) Appl. No.: 18/877,356

(22) PCT Filed: Jul. 3, 2023
(86) PCT No.: PCT/CN2023/105480

§ 371 Date: Dec. 20, 2024

(30) Foreign Application Priority Data
Jul. 6, 2022 (WO) PCT/CN2022/104038

(51) Int. Cl. C07K 16/28; A61K 39/395; A61K 47/12; A61K 47/18; A61K 47/26

(52) U.S. Cl. CPC C07K 16/2827; A61K 39/39591; A61K 47/12; A61K 47/183; A61K 47/26; C07K 16/2878; C07K 2317/31

(57) ABSTRACT

Provided are formulations for anti-PD-L1/anti-4-1BB bispecific antibodies and methods of making and using the same. In one aspect, the disclosure relates to a stable aqueous pharmaceutical formulation comprising an anti-PD-L1/anti-4-1BB bispecific antibody or antigen-binding fragment thereof.

### TECHNICAL FIELD

This application claims the priority of PCT Application No.PCT/CN2022/104038, filed with the China National Intellectual Property Administration on Jul. 6, 2022, and titled with “FORMULATIONS FOR ANTI-PD-L1/4-1BB BISPECIFIC ANTIBODIES”, which is hereby incorporated by reference in its entirety.

This disclosure relates to formulations for anti-PD-L1/anti-4-1BB bispecific antibodies and methods of making and using the same.

### BACKGROUND

Antibodies, as other protein therapeutics, are complex molecules. Usually, large amounts of antibodies have to be used in pharmaceutical formulations due to their therapeutically effective dose in mammals. Because antibodies are larger and more complex than traditional organic and inorganic drugs, the formulation of antibodies poses special problems. For an antibody to remain biologically active, a formulation must preserve the conformational integrity of the amino acid sequence, while at the same time protecting the antibody from degradation. In addition, monoclonal and polyclonal antibodies in particular may be relatively unstable. A large number of formulation options are available, and not one approach or system is suitable for all antibodies. Several factors to be considered have been reported (Wang et al. “Antibody structure, instability, and formulation.” Journal of pharmaceutical sciences 96.1 (2007): 1-26).

Numerous factors may affect an antibody's stability. In fact, the antibody structures may be heterogenous, which further complicates making a stable formulation for these antibodies. In addition, the excipients included in antibody formulations should preferably minimize any potential immune response. There is a need for a stable formulation for this antibody.

### SUMMARY

This disclosure relates to formulations for anti-PD-L1/anti-4-1BB bispecific antibody, particularly BH3120, and methods of making and using the same. BH3120 can binds to PD-L1 and 4-1BB simultaneously, and is used for cancer treatment. Provided herein are BH3120 formulations with a good thermal stability. The formulations described herein can keep the anti-PD-L1/anti-4-BB bispecific antibody in liquid solution that is not easily decomposed, aggregated, or undergone undesired chemical modifications.

In one aspect, the disclosure is related to a stable aqueous pharmaceutical formulation comprising an anti-PD-L1/anti-4-1BB bispecific antibody or antigen-binding fragment thereof, and a buffer, in some embodiments, the formulation has a pH of about 5.0 to about 6.5. In some embodiments, the formulation has a pH of about 5.5 to about 6.0. In some embodiments, the formulation has a pH of about 5.6 or about 5.8.

In some embodiments, the formulation has a pH of about 5.6 to about 5.8.

In some embodiments, the formulation has a pH of about 5.8 to about 6.0.

In some embodiments, the anti-PD-L1/anti-4-1BB bispecific antibody or antigen-binding fragment thereof can bind to a human PD-L1 and/or a human 4-1BB. In some embodiments, the anti-PD-L1/anti-4-1BB bispecific antibody or antigen-binding fragment thereof has a concentration of about 10 mg/ml to about 100 mg/ml. In some embodiments, the anti-PD-L1/anti-4-1BB bispecific antibody or antigen-binding fragment thereof has a concentration of about 20 mg/ml to about 80 mg/ml. In some embodiments, the -PD-L1/anti-4-1BB bispecific antibody or antigen-binding fragment thereof has a concentration of about 50 mg/ml. In some embodiments, the buffer comprises a buffering agent selected from the group consisting of acetic acid, sodium acetate, tartrate, hydrogen chloride, sodium dihydrogen phosphate, and combinations thereof In some embodiments, the buffer comprises acetic acid and/or sodium acetate. In some embodiments, the formulation comprises an acetate acid/sodium acetate buffer with a concentration of about 10 mM to about 100 mM. In some embodiments, the formulation comprises an acetate acid/sodium acetate buffer with a concentration of about 10 or about 15 mM. In some embodiments, the formulation further comprises a tonicity agent selected from the group consisting of trehalose, sucrose, proline, glycine, arginine, alanine, glutamate, methionine, sodium chloride, potassium chloride, magnesium chloride, sodium sulfate and combinations thereof. In some embodiments, the tonicity agent is trehalose, arginine, or a combination thereof In some embodiments, the formulation comprises from about 10 mM to about 200 mM of arginine. In some embodiments, the formulation comprises about 100 mM or about 140 mM of arginine. In some embodiments, the arginine is the hydrochloride salt form of arginine (arginine-HCl). In some embodiments, the formulation comprises from about 10 mM to about 300 mM of trehalose. In some embodiments, the formulation comprises about 130 mM of trehalose. In some embodiments, the formulation further comprises a surfactant selected from the group consisting of polysorbate 20, polysorbate 40, polysorbate 60, polysorbate 80, SDS, poloxamer 188 (Pluronic® F68), and combinations thereof In some embodiments, the surfactant is polysorbate 80. In some embodiments, the formulation comprises from about 0.01 mg/ml to about 10 mg/ml polysorbate 80. In some embodiments, the formulation comprises about 0.2 mg/ml polysorbate 80.

In one aspect, the disclosure is related to a stable aqueous pharmaceutical formulation comprising: an anti-PD-L1/anti-4-1BB bispecific antibody or antigen-binding fragment thereof with a concentration of about 10 mg/ml to about 100 mg/ml; a tonicity agent with a concentration of about 100 mM to about 300 mM; a surfactant with a concentration of about 0.01 mg/ml-about 10 mg/ml; and a buffer system, in some embodiments, the formulation has a pH of about 5.0 to about 6.5. In some embodiments, the formulation has a pH of about 5.5 to about 6.0. In some embodiments, the buffer system comprises one or more buffering agents selected from the group consisting of acetic acid, sodium acetate, tartrate, hydrogen chloride, and sodium dihydrogen phosphate. In some embodiments, the buffer system comprises acetic acid/sodium acetate buffer with a concentration of about 10 to about 100 mM. In some embodiments, the tonicity agent is trehalose, sucrose, proline, glycine, arginine, alanine, glutamate, methionine, sodium chloride, potassium chloride, magnesium chloride, sodium sulfate, or combinations thereof. In some embodiments, the tonicity agent is trehalose, arginine (e.g., arginine-HCl), or a combination thereof. In some embodiments, the surfactant is polysorbate 20, polysorbate 40, polysorbate 60, polysorbate 80, SDS, poloxamer 188 (Pluronic® F68), or combinations thereof. In some embodiments, the surfactant is polysorbate 80.

In one aspect, the disclosure is related to a stable aqueous pharmaceutical formulation comprising or consisting of an anti-PD-L1/anti-4-1BB bispecific antibody or antigen-binding fragment thereof with a concentration of about 10 mg/ml to about 100 mg/ml; arginine (e.g., arginine-HC1) with a concentration of about 10 mM to about 200 mM; optionally trehalose with a concentration of about 10 mM to about 300 mM; polysorbate 80 with a concentration of about 0.01 mg/ml to about 10 mg/ml; and acetic acid/sodium acetate buffer with a concentration of about 10 to about 100 mM, in some embodiments, the formulation has a pH of about 5.5 to about 6.0. In some embodiments, the concentration of the anti-PD-L1/anti-4-1BB bispecific antibody or antigen-binding fragment thereof is about 20 mg/ml, about 50 mg/ml, or about 80 mg/ml. In some embodiments, the concentration of arginine is about 100 mM or about 140 mM. In some embodiments, the concentration of trehalose is about 130 mM. In some embodiments, the concentration of polysorbate 80 is about 0.2 mg/ml. In some embodiments, the concentration of acetic acid/sodium acetate buffer is about 10 mM or about 15 mM.

In some embodiments, the anti-PD-L1/anti-4-1BB bispecific antibody or antigen-binding fragment thereof comprises: a first heavy chain variable region (VH1) comprising complementarity determining regions (CDRs) 1, 2, and 3, in some embodiments, the VH1 CDR1 region comprises an amino acid sequence that is at least 80% identical to SEQ ID NO:15, the VH1 CDR2 region comprises an amino acid sequence that is at least 80% identical to SEQ ID NO:16, and the VH1 CDR3 region comprises an amino acid sequence that is at least 80% identical to SEQ ID NO:17; a first light chain variable region (VL1) comprising CDRs 1, 2, and 3, in some embodiments, the VL1 CDR1 region comprises an amino acid sequence that is at least 80% identical to SEQ ID NO:18, the VL1 CDR2 region comprises an amino acid sequence that is at least 80% identical to SEQ ID NO:19, and the VL1 CDR3 region comprises an amino acid sequence that is at least 80% identical to SEQ ID NO:20, in some embodiments, the VH1 and VL1 can interact with each other, forming an antigen-binding site that binds to PD-L1.

In some embodiments, the anti-PD-L1/anti-4-1BB bispecific antibody or antigen-binding fragment thereof comprises: a second heavy chain variable region (VH2) comprising complementarity determining regions (CDRs) 1, 2, and 3, in some embodiments, the VH2 CDR1 region comprises an amino acid sequence that is at least 80% identical to SEQ ID NO:21, the VH2 CDR2 region comprises an amino acid sequence that is at least 80% identical to SEQ ID NO:22, and the VH2 CDR3 region comprises an amino acid sequence that is at least 80% identical to SEQ ID NO:23; and a second light chain variable region (VL2) comprising CDRs 1, 2, and 3, in some embodiments, the VL2 CDR1 region comprises an amino acid sequence that is at least 80% identical to SEQ ID NO:24, the VL2 CDR2 region comprises an amino acid sequence that is at least 80% identical to SEQ ID NO:25, and the VL2 CDR3 region comprises an amino acid sequence that is at least 80% identical to SEQ ID NO:26, in some embodiments, the VH2 and VL2 can interact with each other, forming an antigen-binding site that binds to 4-1BB.

In some embodiments, the anti-PD-L1/anti-4-1BB bispecific antibody or antigen-binding fragment thereof comprises: a first heavy chain variable region (VH1) comprising an amino acid sequence that is at least 90% identical to SEQ ID NO:6, and a first light chain variable region (VL1) comprising an amino acid sequence that is at least 90% identical to SEQ ID NO:2, in some embodiments, the VH1 and VL1 can interact with each other, forming an antigen-binding site that binds to PD-L1, and a second heavy chain variable region (VH2) comprising an amino acid sequence that is at least 90% identical to SEQ ID NO:12, and a second light chain variable region (VL2) comprising an amino acid sequence that is at least 90% identical to SEQ ID NO:10, in some embodiments, the VH2 and VL2 can interact with each other, forming an antigen-binding site that binds to 4-1BB.

In some embodiments, the anti-PD-L1/anti-4-1BB bispecific antibody or antigen-binding fragment thereof comprises: a first heavy chain constant region (CH1) comprising an amino acid sequence that is at least 90% identical to SEQ ID NO:8, and a first light chain constant region (CL1) comprising an amino acid sequence that is at least 90% identical to SEQ ID NO:4, in some embodiments, the CH1 and CL1 can interact with each other, forming an antigen-binding site that binds to PD-L1, and a second heavy chain variable region (CH2) comprising an amino acid sequence that is at least 90% identical to SEQ ID NO:14, and a second light chain variable region (CL2) comprising an amino acid sequence that is at least 90% identical to SEQ ID NO:4, in some embodiments, the CH2 and CL2 can interact with each other, forming an antigen-binding site that binds to 4-1BB.

In some embodiments, the anti-PD-L1/anti-4-1BB bispecific antibody or antigen-binding fragment thereof comprises: a first heavy chain comprising an amino acid sequence that is at least 90% identical to SEQ ID NO:27, and a first light chain comprising an amino acid sequence that is at least 90% identical to SEQ ID NO:28, in some embodiments, the first heavy chain and the first light chain can interact with each other and bind to PD-L1; and a second heavy chain comprising an amino acid sequence that is at least 90% identical to SEQ ID NO:29, and a second light chain comprising an amino acid sequence that is at least 90% identical to SEQ ID NO:30, in some embodiments, the second heavy chain and the second light chain can interact with each other and bind to 4-1BB.

In some embodiments, the formulation has long-term stability.

In one aspect, the disclosure is related to a method of treating a subject having cancer, the method comprising administering a therapeutically effective amount of the formulation as described herein to the subject. In some embodiments, the subject has leukemia, lymphoma, myeloma, brain tumor, head and neck squamous cell cancer, non-small cell lung cancer, nasopharyngeal cancer, esophageal cancer, gastric cancer, pancreatic cancer, gallbladder cancer, liver cancer, colorectal cancer, breast cancer, ovarian cancer, cervical cancer, endometrial cancer, uterine sarcoma, prostate cancer, bladder cancer, renal cell cancer, melanoma, small cell lung cancer or bone cancer. In some embodiments, the subject is a human.

In one aspect, the disclosure is related to a formulation of an anti-PD-L1/anti-4-1BB bispecific antibody or antigen-binding fragment thereof exhibiting long term stability comprising, consisting of, or consisting essentially of. (a) an anti-PD-L1/anti-4-1BB bispecific antibody or antigen-binding fragment thereof comprising an anti-PD-L1 arm comprising a first heavy chain variable region comprising an amino acid sequence that is at least 90% identical to SEQ ID NO:6 and a first light chain variable region comprising an amino acid sequence that is at least 90% identical to SEQ ID NO:2; and an anti-4-1BB arm comprising a second heavy chain variable region comprising an amino acid sequence that is at least 90% identical to SEQ ID NO:12 and a second light chain variable region comprising an amino acid sequence that is at least 90% identical to SEQ ID NO:10; (b) 10 mM or 15 mM acetic acid/sodium acetate; (c) 100 mM or 140 mM arginine (e.g., arginine-HC1); (d) optionally 130 mM trehalose; and (e) 0.2 mg/ml polysorbate 80, in some embodiments, the formulation has a pH of 5.5-6.0 (e.g., 5.6 or 5.8). In some embodiments, the anti-PD-L1/anti-4-1BB bispecific antibody or antigen-binding fragment thereof has a concentration of about 10 mg/ml to about 100 mg/ml. In some embodiments, the anti-PD-L1/anti-4-1BB bispecific antibody or antigen-binding fragment thereof has a concentration of about 20 mg/ml to about 80 mg/ml. In some embodiments, the anti-PD-L1/anti-4-1BB bispecific antibody or antigen-binding fragment thereof has a concentration of about 20 mg/ml, about 50 mg/ml, or about 100 mg/ml.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include, e.g., monoclonal antibodies, polyclonal antibodies, multi-specific antibodies (e.g., bi-specific antibodies), single-chain antibodies, chimeric antibodies, human antibodies, mouse antibodies, etc. In some embodiments, an antibody can contain an Fc region of a mouse antibody, or a human antibody. The term antibody also includes derivatives, e.g., bi-specific antibodies, single-chain antibodies, diabodies, linear antibodies, and multi-specific antibodies formed from antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain). Non-limiting examples of antibody fragments include, e.g., Fab, Fab′, F(ab′)₂, and Fv fragments.

The monoclonal antibodies herein specifically include “chimeric” antibodies. As used herein, the term “chimeric antibody” refers to an antibody (immuno globulin) in which a portion of the heavy and/or light chain is identical with or homologous to corresponding sequences in antibodies derived from a particular species, while the remainder of the chain(s) is identical with or homologous to corresponding sequences in antibodies derived from another species, as well as fragments of such antibodies, so long as they exhibit the desired biological activity. Typically, chimeric antibodies are antibodies whose light and heavy chain genes have been constructed, e.g., by genetic engineering, from antibody variable and constant region genes belonging to different species. For example, the variable segments of the genes from a mouse monoclonal antibody can be joined to human constant segments. In some embodiments, the antigen binding site is derived from mouse while the Fc portion is human.

As used herein, the term “single-chain antibody” refers to a single polypeptide that contains at least two immunoglobulin variable domains (e.g., a variable domain of a mammalian immunoglobulin heavy chain or light chain) that is capable of specifically binding to an antigen. Non-limiting examples of single-chain antibodies are described herein.

As used herein, the terms “subject” and “patient” are used interchangeably throughout the specification and describe an animal, human or non-human, to whom treatment according to the methods of the present invention is provided. Veterinary and non-veterinary applications are contemplated by the present invention. In some embodiments, the subject is a mammal (e.g., a non-human mammal). The subjects can include but are not limited to mice, rats, hamsters, guinea-pigs, rabbits, ferrets, cats, dogs, giant panda, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals. In some embodiments, the subject is a human.

As used herein, when referring to an antibody, the phrases “specifically binding” and “specifically binds” mean that the antibody interacts with its target molecule (e.g., PD-L1 or 4-1BB) preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to a PD-L1 molecule may be referred to as a PD-L1-specific antibody or an anti-PD-L1 antibody.

As used herein, the term “about” or “approximately” shall generally mean within 20 percent, within 10 percent, within 5, 4, 3, 2 or 1 percent of a given value or range. Numerical quantities given are approximate, meaning that the term “around,” “about” or “approximately” can be inferred if not expressly stated.

As used herein, the term “sugar” refers to monosaccharides, disachharides, and polysaccharides. Examples of sugars include, but are not limited to, sucrose, glucose, dextrose, trehalose, and others.

As used herein, the term “long-term storage” or “long term stability” is understood to mean that the pharmaceutical composition can be stored for three months or more, for six months or more, and preferably for one year or more, most preferably a minimum stable shelf life of at least two years.

As used herein, the term “stable” with respect to long-term storage is understood to mean that the antibody contained in the pharmaceutical compositions does not lose more than 30%, preferably 20%, or more preferably 15%, or even more preferably 10%, and most preferably 5% of its activity relative to activity of the composition at the beginning of storage.

As used herein, the term “substantially free” means that either no substance is present or only minimal, trace amounts of the substance are present which do not have any substantial impact on the properties of the composition. If reference is made to no amount of a substance, it should be understood as “no detectable amount”.

As used herein, the term “pharmaceutically acceptable carrier” refers to a non-toxic solid, semisolid or liquid filler, diluent, encapsulating material, formulation auxiliary, or excipient of any conventional type. A pharmaceutically acceptable carrier is non-toxic to recipients at the dosages and concentrations employed and is compatible with other ingredients of the formulation.

As used herein, the terms “pharmaceutical composition” and “formulation” are used interchangeably.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DETAILED DESCRIPTION

Antibodies, like other proteins, are prone to a variety of physical and chemical degradations. Antibody instabilities can be observed in liquid, frozen, and lyophilized states. In many cases, multiple degradation pathways can occur at the same time and the degradation mechanism may change depending on the stress conditions. These degradation pathways are divided into two major categories—physical and chemical instabilities.

Despite antibodies have similar structures, the behavior of antibodies seems to vary. This disclosure provides formulations for anti-PD-L1/anti-4-1BB bispecific antibodies and methods of making and using the same. In one aspect, the disclosure provides stable aqueous formulations that allow the long term storage of anti-PD-L1/anti-4-1BB antibodies.

### Antibody Formulation

This disclosure relates to formulations for anti-PD-L1/anti-4-1BB bispecific antibodies. Formulations of the present disclosure can be found in the form of liquids and lyophilized powders that comprise anti-PD-L1/anti-4-1BB bispecific antibodies or antigen binding fragments thereof Further, such formulations can include buffering agents, tonicity agents, surfactants, stabilizing agents, and some other excipients.

In some embodiments, the anti-PD-L1/anti-4-1BB bispecific antibodies or antigen binding fragments thereof can be any anti-PD-L1/anti-4-1BB bispecific antibody or antigen-binding fragment as described herein.

In some embodiments, the concentration for the anti-PD-L1/anti-4-1BB bispecific antibodies as described herein in the formulation is about or at least 1, 2, 3, 4, 5, 6, 7, 8, 9, 10, 20, 30, 40, 50, 60, 70, 80, 90, or 100 mg/ml. In some embodiments, the concentration is no more than 50, 60, 70, 80, 90, 100 or 200 mg/ml. In some embodiments, the antibody or the antigen-binding fragment thereof is present in the formulations in an amount from about 5 mg/mL to about 200 mg/mL, about 5 mg/mL to about 180 mg/mL, about 5 mg/mL to about 160 mg/mL, about 5 mg/mL to about 140 mg/mL, about 5 mg/mL to about 120 mg/mL, about 5 mg/mL to about 100 mg/mL, about 5 mg/mL to about 80 mg/mL, about 5 mg/mL to about 60 mg/mL, about 5 mg/mL to about 40 mg/mL, about 5 mg/mL to about 20 mg/mL, about 10 mg/mL to about 200 mg/mL, about 10 mg/mL to about 180 mg/mL, about 10 mg/mL to about 160 mg/mL, about 10 mg/mL to about 140 mg/mL, about 10 mg/mL to about 120 mg/mL, about 10 mg/mL to about 100 mg/mL, about 10 mg/mL to about 80 mg/mL, about 10 mg/mL to about 60 mg/mL, about 10 mg/mL to about 40 mg/mL, about 10 mg/mL to about 20 mg/mL, about 20 mg/mL to about 200 mg/mL, about 20 mg/mL to about 180 mg/mL, about 20 mg/mL to about 160 mg/mL, about 20 mg/mL to about 140 mg/mL, about 20 mg/mL to about 120 mg/mL, about 20 mg/mL to about 100 mg/mL, about 20 mg/mL to about 80 mg/mL, about 20 mg/mL to about 60 mg/mL, about 20 mg/mL to about 40 mg/mL, about 20 mg/mL to about 30 mg/mL, about 30 mg/mL to about 200 mg/mL, about 30 mg/mL to about 180 mg/mL, about 30 mg/mL to about 160 mg/mL, about 30 mg/mL to about 140 mg/mL, about 30 mg/mL to about 120 mg/mL, about 30 mg/mL to about 100 mg/mL, about 30 mg/mL to about 80 mg/mL, about 30 mg/mL to about 60 mg/mL, about 30 mg/mL to about 40 mg/mL, about 40 mg/mL to about 200 mg/mL, about 40 mg/mL to about 180 mg/mL, about 40 mg/mL to about 160 mg/mL, about 40 mg/mL to about 140 mg/mL, about 40 mg/mL to about 120 mg/mL, about 40 mg/mL to about 100 mg/mL, about 40 mg/mL to about 80 mg/mL, about 40 mg/mL to about 60 mg/mL, about 40 mg/mL to about 50 mg/mL, about 50 mg/mL to about 200 mg/mL, about 50 mg/mL to about 180 mg/mL, about 50 mg/mL to about 160 mg/mL, about 50 mg/mL to about 140 mg/mL, about 50 mg/mL to about 120 mg/mL, about 50 mg/mL to about 100 mg/mL, about 50 mg/mL to about 80 mg/mL, about 50 mg/mL to about 60 mg/mL, about 60 mg/mL to about 200 mg/mL, about 60 mg/mL to about 180 mg/mL, about 60 mg/mL to about 160 mg/mL, about 60 mg/mL to about 140 mg/mL, about 60 mg/mL to about 120 mg/mL, about 60 mg/mL to about 100 mg/mL, about 60 mg/mL to about 80 mg/mL, about 60 mg/mL to about 70 mg/mL, about 70 mg/mL to about 200 mg/mL, about 70 mg/mL to about 180 mg/mL, about 70 mg/mL to about 160 mg/mL, about 70 mg/mL to about 140 mg/mL, about 70 mg/mL to about 120 mg/mL, about 70 mg/mL to about 100 mg/mL, about 70 mg/mL to about 80 mg/mL, about 80 mg/mL to about 200 mg/mL, about 80 mg/mL to about 180 mg/mL, about 80 mg/mL to about 160 mg/mL, about 80 mg/mL to about 140 mg/mL, about 80 mg/mL to about 120 mg/mL, about 80 mg/mL to about 100 mg/mL, about 80 mg/mL to about 90 mg/mL, about 90 mg/mL to about 200 mg/mL, about 90 mg/mL to about 180 mg/mL, about 90 mg/mL to about 160 mg/mL, about 90 mg/mL to about 140 mg/mL, about 90 mg/mL to about 120 mg/mL, about 90 mg/mL to about 100 mg/mL, about 100 mg/mL to about 200 mg/mL, about 100 mg/mL to about 180 mg/mL, about 100 mg/mL to about 160 mg/mL, about 100 mg/mL to about 140 mg/mL, about 100 mg/mL to about 120 mg/mL, about 120 mg/mL to about 200 mg/mL, about 120 mg/mL to about 180 mg/mL, about 120 mg/mL to about 160 mg/mL, about 120 mg/mL to about 140 mg/mL, about 140 mg/mL to about 200 mg/mL, about 140 mg/mL to about 180 mg/mL, about 140 mg/mL to about 160 mg/mL, about 160 mg/mL to about 200 mg/mL, about 160 mg/mL to about 180 mg/mL, or about 180 mg/mL to about 200 mg/mL. In some embodiments, the concentration is about 50 mg/mL to about 500 mg/mL, about 25 mg/mL to about 400 mg/mL, about 50 mg/mL to about 250 mg/mL, about 5 mg/mL to about 280 mg/mL, about 5 mg/mL to about 200 mg/mL, about 5 mg/mL to about 125 mg/mL, about 5 mg/mL to about 75 mg/mL, about 5 mg/mL to about 50 mg/mL, or about 5 mg/mL to about 25 mg/mL. In some embodiments, the concentration is about 1 to about 100 mg/ml, about 5 to about 100 mg/ml, about 10 to about 100 mg/ml, about 20 to about 100 mg/ml, about 30 to about 100 mg/ml, about 40 to about 100 mg/ml, about 50 to about 100 mg/ml, or about 10 to about 50 mg/ml. For example, the antibody or antigen binding fragment can be present in the formulation in an amount of about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, about 125 mg/mL, about 130 mg/mL, about 135 mg/mL, about 140 mg/mL, about 145 mg/mL, about 150 mg/mL, about 155 mg/mL, about 160 mg/mL, about 165 mg/mL, about 170 mg/mL, about 175 mg/mL, about 180 mg/mL, about 185 mg/mL, about 190 mg/mL, about 195 mg/mL, about 200 mg/mL, about 205 mg/mL, about 210 mg/mL, about 215 mg/mL, about 220 mg/mL, about 225 mg/mL, about 230 mg/mL, about 235 mg/mL, about 240 mg/mL, about 245 mg/mL, or about 250 mg/mL.

In alternative embodiments, the antibody or antigen binding fragment can be present in the formulation in an amount from about 5 to about 10 mg/mL, from about 11 to about 20 mg/mL, from about 21 to about 30 mg/mL, from about 31 to about 40 mg/mL, from about 41 to about 50 mg/mL, from about 51 to about 75 mg/mL, from about 76 to about 100 mg/mL, from about 101 to about 125 mg/mL, from about 126 to about 150 mg/mL, from about 151 to about 175 mg/mL, from about 176 to about 200 mg/mL, from about 201 mg/mL to about 225 mg/mL, from about 226 mg/mL to about 250 mg/mL, from about 5 to about 30 mg/mL, from about 10 to about 50 mg/mL, from about 10 to about 20 mg/mL, or from about 10 to about 30 mg/mL. In some embodiments, the concentration is 20, 50, or 80 mg/ml.

As the antibody has a relatively high concentration in the formulation, formulations for the anti-PD-L1/anti-4-1BB bispecific antibodies are developed to improve the stability of these antibodies in the formulation.

### Buffering Agents

The formulations can include various buffering agents, e.g., acetic acid, sodium acetate, tartrate, sodium dihydrogen phosphate, and combinations thereof. A buffering agent maintains a physiologically suitable pH. In addition, a buffering agent enhances isotonicity and chemical stability of the formulation.

In some embodiments, the buffering agent is present in the formulations at a concentration from about 10 mM to about 100 mM, e.g., about 10 mM to about 15 mM. For example, the buffering agent can be present in the formulation at a concentration about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, about 50 mM, about 51 mM, about 52 mM, about 53 mM, about 54 mM, about 55 mM, about 56 mM, about 57 mM, about 58 mM, about 59 mM, about 60 mM, about 61 mM, about 62 mM, about 63 mM, about 64 mM, about 65 mM, about 66 mM, about 67 mM, about 68 mM, about 69 mM, about 70 mM, about 71 mM, about 72 mM, about 73 mM, about 74 mM, about 75 mM, about 76 mM, about 77 mM, about 78 mM, about 79 mM, about 80 mM, about 81 mM, about 82 mM, about 83 mM, about 84 mM, about 85 mM, about 86 mM, about 87 mM, about 88 mM, about 89 mM, about 90 mM, about 91 mM, about 92 mM, abou 93 mM, about 94 mM, about 95 mM, about 96 mM, about 97 mM, about 98 mM, about 99 mM, or about 100 mM.

In certain embodiments, the formulations have a pH at or below pH 6.5. In certain embodiments, the formulations have a pH at or below pH 6.0. In certain embodiments, the formulations have a pH at or above pH 5.0. In certain embodiments, the formulations have a pH at or above pH 5.5.

In some embodiments, the formulations have a pH of about 5.0 to about 6.5. In some embodiments, the pH is about 5.0 to about 6.4, about 5.0 to about 6.3, about 5.0 to about 6.2, about 5.0 to about 6.1, about 5.0 to about 6.0, about 5.0 to about 5.9, about 5.0 to about 5.8, about 5.0 to about 5.7, about 5.0 to about 5.6, about 5.0 to about 5.5, about 5.0 to about 5.4, about 5.0 to about 5.3, about 5.0 to about 5.2, about 5.0 to about 5.1, about 5.1 to about 6.5, about 5.1 to about 6.4, about 5.1 to about 6.3, about 5.1 to about 6.2, about 5.1 to about 6.1, about 5.1 to about 6.0, about 5.1 to about 5.9, about 5.1 to about 5.8, about 5.1 to about 5.7, about 5.1 to about 5.6, about 5.1 to about 5.5, about 5.1 to about 5.4, about 5.1 to about 5.3, about 5.1 to about 5.2, about 5.2 to about 6.5, about 5.2 to about 6.4, about 5.2 to about 6.3, about 5.2 to about 6.2, about 5.2 to about 6.1, about 5.2 to about 6.0, about 5.2 to about 5.9, about 5.2 to about 5.8, about 5.2 to about 5.7, about 5.2 to about 5.6, about 5.2 to about 5.5, about 5.2 to about 5.4, about 5.2 to about 5.3, about 5.3 to about 6.5, about 5.3 to about 6.4, about 5.3 to about 6.3, about 5.3 to about 6.2, about 5.3 to about 6.1, about 5.3 to about 6.0, about 5.3 to about 5.9, about 5.3 to about 5.8, about 5.3 to about 5.7, about 5.3 to about 5.6, about 5.3 to about 5.5, about 5.3 to about 5.4, about 5.4 to about 6.5, about 5.4 to about 6.4, about 5.4 to about 6.3, about 5.4 to about 6.2, about 5.4 to about 6.1, about 5.4 to about 6.0, about 5.4 to about 5.9, about 5.4 to about 5.8, about 5.4 to about 5.7, about 5.4 to about 5.6, about 5.4 to about 5.5, about 5.5 to about 6.5, about 5.5 to about 6.4, about 5.5 to about 6.3, about 5.5 to about 6.2, about 5.5 to about 6.1, about 5.5 to about 6.0, about 5.5 to about 5.9, about 5.5 to about 5.8, about 5.5 to about 5.7, about 5.5 to about 5.6, about 5.6 to about 6.5, about 5.6 to about 6.4, about 5.6 to about 6.3, about 5.6 to about 6.2, about 5.6 to about 6.1, about 5.6 to about 6.0, about 5.6 to about 5.9, about 5.6 to about 5.8, about 5.6 to about 5.7, about 5.7 to about 6.5, about 5.7 to about 6.4, about 5.7 to about 6.3, about 5.7 to about 6.2, about 5.7 to about 6.1, about 5.7 to about 6.0, about 5.7 to about 5.9, about 5.7 to about 5.8, about 5.8 to about 6.5, about 5.8 to about 6.4, about 5.8 to about 6.3, about 5.8 to about 6.2, about 5.8 to about 6.1, about 5.8 to about 6.0, about 5.8 to about 5.9, about 5.9 to about 6.5, about 5.9 to about 6.4, about 5.9 to about 6.3, about 5.9 to about 6.2, about 5.9 to about 6.1, about 5.9 to about 6.0, about 6.0 to about 6.5, about 6.0 to about 6.4, about 6.0 to about 6.3, about 6.0 to about 6.2, about 6.0 to about 6.1, about 6.1 to about 6.5, about 6.1 to about 6.4, about 6.1 to about 6.3, about 6.1 to about 6.2, about 6.2 to about 6.5, about 6.2 to about 6.4, about 6.2 to about 6.3, about 6.3 to about 6.5, about 6.3 to about 6.4, or about 6.4 to about 6.5. In some embodiments, the pH is about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, or about 6.5.

The pH of the formulation can be measured by any means known to those of skill in the art. A means for measuring pH is using a pH meter with a micro-electrode. The pH of the formulation can be adjusted using any means known in the art. Exemplary chemicals for altering the pH of the formulations are hydrochloric acid (HCl) and sodium hydroxide (NaOH).

In some embodiments, the acetate buffer described herein is acetic acid/sodium acetate buffer. In some embodiments, the buffer is acetic acid/sodium acetate buffer. In some embodiments, the buffer described herein includes about 5-100 mM (e.g., about 5-90 mM, about 5-80 mM, about 5-70 mM, about 5-60 mM, about 5-50 mM, about 5-40 mM, about 5-30 mM, about 5-20 mM, about 5-10 mM, about 10-90 mM, about 10-80 mM, about 10-70 mM, about 10-60 mM, about 10-50 mM, about 10-40 mM, about 10-30 mM, about 10-20 mM, about 20-100 mM, about 20-90 mM, about 20-80 mM, about 20-70 mM, about 20-60 mM, about 20-50 mM, about 20-40 mM, about 20-30 mM, about 30-100 mM, about 30-90 mM, about 30-80 mM, about 30-70 mM, about 30-60 mM, about 30-50 mM, about 30-40 mM, about 40-100 mM, about 40-90 mM, about 40-80 mM, about 40-70 mM, about 40-60 mM, about 40-50 mM, about 50-100 mM, about 50-90 mM, about 50-80 mM, about 50-70 mM, about 50-60 mM, about 60-100 mM, about 60-90 mM, about 60-80 mM, about 60-70 mM, about 70-100 mM, about 70-90 mM, about 70-80 mM, about 80-100 mM, about 80-90 mM, or about 90-100 mM) acetic acid/sodium acetate. When referring to the concentration of acetic acid/sodium acetate buffer, the concentration means the number of moles of acetic acid and sodium acetate together divided by volume. In some embodiments, the buffer described herein includes about 10 mM, about 15 mM, about 20 mM, about 30 mM, about 40 mM, about 50 mM, about 60 mM, about 70 mM, about 80 mM, about 90 mM, or about 100 mM acetic acid/sodium acetate. In some embodiments, the formulation is substantially free from any other buffer (e.g., a citrate buffer, succinate buffer, histidine buffer, or phosphate buffer).

As shown in the present disclosure, the formulations as described herein provide significant improvements on the stability of anti-PD-L1/anti-4-1BB bispecific antibodies as described herein. For example, when a contemplated formulation includes an anti-PD-L1/anti-4-1BB bispecific antibody and an acetic acid/sodium acetate buffer, wherein the pH of the formulation is within 5.0-6.5 (e.g., 5.6 or 5.8), the formulations provide significant improvements in the stability, wherein no visible foreign matters can be observed.

In some embodiments, the formulation described herein includes an anti-PD-L1/anti-4-1BB bispecific antibody (10-100 mg/ml), 10-100 mM acetic acid/sodium acetate, 10-200 mM arginine-HC1, 0.01 mg/ml-10 mg/ml polysorbate 80, with a pH 5.5-6.0. In some embodiments, the formulation further includes 100-300 mM trehalose.

In some embodiments, the formulation described herein includes an anti-PD-L1/anti-4-1BB bispecific antibody, 10-20 mM acetic acid/sodium acetate, 100-200 mM arginine-HC1, 0.1 mg/ml-0.5 mg/ml polysorbate 80, with a pH 5.5-6.0. In some embodiments, the formulation further includes 100-150 mM trehalose.

### Surfactants

The formulations can, optionally, comprise a surfactant. A surfactant can be a stabilizing agent. Surfactants/stabilizing agents are chemical compounds that interact and stabilize biological molecules and/or general pharmaceutical excipients in a formulation. Surfactants generally protect the active agent from air/solution interface induced stresses and solution/surface induced stresses, which may otherwise result in protein aggregation. Surfactants can include, but are not limited to, polysorbates, glycerin, dicarboxylic acids, oxalic acid, succinic acid, fumaric acids, phthalic acids, and combinations thereof. Surfactants, including e.g. non-ionic or ionic detergents, can be used as long as they are pharmaceutically acceptable, i.e., suitable for administration to subjects. In some embodiments, the surfactant is a polysorbate. Examples of polysorbates include polysorbate 20, polysorbate 40, polysorbate 60, polysorbate 65, and polysorbate 80.

In some embodiments, the surfactant is present in the formulations in an amount from about 0.01 mg/ml to about 1 mg/ml. For example, the surfactant can be present in the formulations in an amount of about 0.01 mg/ml, about 0.02 mg/ml, about 0.03 mg/ml, about 0.04 mg/ml, about 0.05 mg/ml, about 0.06 mg/ml, about 0.07 mg/ml, about 0.08 mg/ml, about 0.09 mg/ml, about 0.1 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml, and about 1 mg/ml. In particular embodiments, the surfactant is present in the formulations from about 0.03 mg/ml to about 0.5 mg/ml, about 0.04 mg/ml to about 0.25 mg/ml, or about 0.1 mg/ml to about 0.2 mg/ml, e.g., about 0.2 mg/ml.

In some embodiments, the surfactant is polysorbate 20 or polysorbate 80. For example, polysorbate 80 can be present in an amount from about 0.01 mg/ml to about 1 mg/ml, about 0.1 mg/ml to about 0.5 mg/ml, about 0.1 mg/ml to about 0.3 mg/ml, e.g., about 0.2 mg/ml. In alternative embodiments, polysorbate 80 is present in an amount from about 0.01 mg/ml to about 1 mg/ml, about 0.05 mg/ml to about 0.5 mg/ml, and about 0.1 mg/ml to about 0.2 mg/ml. In further alternative embodiments, polysorbate 80 is present in an amount from about 0.01 mg/ml to about 1 mg/ml, about 0.05 mg/ml to about 0.5 mg/ml, and about 0.1 mg/ml to about 0.3 mg/ml, e.g., about 0.2 mg/ml. In some embodiments, the formulation described herein includes about 0.01 mg/ml to about 10 mg/ml (e.g., about 0.01 mg/ml to about 9 mg/ml, about 0.01 mg/ml to about 8 mg/ml, about 0.01 mg/ml-about 7 mg/ml, about 0.01 mg/ml to about 6 mg/ml, about 0.01 mg/ml to about 5 mg/ml, about 0.01 mg/ml to about 4 mg/ml, about 0.01 mg/ml to about 3 mg/ml, about 0.01 mg/ml to about 2 mg/ml, about 0.01 mg/ml to about 1 mg/ml, about 0.01 mg/ml to about 0.9 mg/ml, about 0.01 mg/ml to about 0.8 mg/ml, about 0.01 mg/ml to about 0.7 mg/ml, about 0.01 mg/ml to about 0.6 mg/ml, about 0.01 mg/ml to about 0.5 mg/ml, about 0.01 mg/ml to about 0.4 mg/ml, about 0.01 mg/ml to about 0.3 mg/ml, about 0.01 mg/ml to about 0.2 mg/ml) polysorbate 80.

Polysorbate 20 can be present in an amount from about 0.01 mg/ml to about 1 mg/ml, about 0.1 mg/ml to about 0.5 mg/ml, about 0.1 mg/ml to about 0.3 mg/ml, e.g., about 0.2 mg/ml. In alternative embodiments, polysorbate 20 is present in an amount from about 0.01 mg/ml to about 1 mg/ml, about 0.05 mg/ml to about 0.5 mg/ml, and about 0.1 mg/ml to about 0.2 mg/ml. In further alternative embodiments, polysorbate 20 is present in an amount from about 0.01 mg/ml to about 1 mg/ml, about 0.05 mg/ml to about 0.5 mg/ml, and about 0.1 mg/ml to about 0.3 mg/ml, e.g., about 0.2 mg/ml.

### Tonicity Agents

The formulations of the invention can further comprise a tonicity agent. Typically, tonicity agents are used to adjust or maintain the osmolality of the formulations in order to bring it closer to the osmotic pressure of body fluids, such as blood or plasma. Tonicity agents can also maintain the antibody or antigen binding fragment levels in a formulation. As used herein, the term “tonicity” refers to the behavior of biologic components in a fluid environment or solution. Isotonic solutions possess the same osmotic pressure as blood plasma, and can be intravenously infused into a subject without changing the osmotic pressure of the subject's blood plasma. Indeed, in certain embodiments, the tonicity agent is present in an amount sufficient to render the formulation suitable for intravenous infusion. Often, the tonicity agent serves as a bulking agent or a stabilizing agent as well. As such, the tonicity agent can allow the antibody or antigen binding fragment thereof to overcome various stresses, such as freezing and shear. Tonicity agents can include, but are not limited to, saccharides, sugars, glycerol, sorbitol, mannitol, sodium chloride, potassium chloride, magnesium chloride, and other inorganic salts. Those skilled in the art are aware that other tonicity agents can be used as long as they are pharmaceutically acceptable, ie., suitable for administration to subjects.

In certain embodiments, the tonicity agent is a saccharide. Examples of saccharides include glucose, sucrose, maltose, trehalose, dextrose, xylitol, fructose and mannitol. In some embodiments, the formulation described herein includes about 10-500 mM (e.g., about 10-450 mM, about 10-400 mM, about 10-350 mM, about 10-300 mM, about 10-250 mM, about 10-200 mM, about 10-150 mM, about 10-100 mM, about 10-50 mM, about 50-500 mM, about 50-450 mM, about 50-400 mM, about 50-350 mM, about 50-300 mM, about 50-250 mM, about 50-200 mM, about 50-150 mM, about 50-100 mM, about 100-500 mM, about 100-450 mM, about 100-400 mM, about 100-350 mM, about 100-300 mM, about 100-250 mM, about 100-200 mM, about 100-150 mM, about 150-500 mM, about 150-450 mM, about 150-400 mM, about 150-400 mM, about 150-350 mM, about 150-300 mM, about 150-250 mM, about 150-200 mM, about 200-500 mM, about 200-450 mM, about 200-400 mM, about 200-350 mM, about 200-300 mM, about 200-250 mM, about 250-500 mM, about 250-450 mM, about 250-400 mM, about 250-350 mM, about 250-300 mM, about 300-500 mM, about 300-450 mM, about 300-400 mM, about 300-350 mM, about 350-500 mM, about 350-450 mM, about 350-400 mM, about 400-500 mM, about 400-450 mM, or about 450-500 mM) of a tonicity agent.

In some embodiments, the tonicity agent is sucrose or trehalose. In some embodiments, the tonicity agent is sucrose. In some embodiments, trehalose has a concentration of about 10-300 mM (e.g., about 10-50 mM, about 50-100 mM, about 100-110 mM, about 110-120 mM, about 120-130 mM, about 130-140 mM, about 140-150 mM, about 150-160 mM, about 160-170 mM, about 170-180 mM, about 180-190 mM, about 190-200 mM, about 210-220 mM, about 220-230 mM, about 230-240 mM, about 240-250 mM, about 250-260 mM, about 260-270 mM, about 270-280 mM, about 280-290 mM, or about 290-300 mM).

In some embodiments, the formulation described herein includes about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, or about 300 mM of trehalose.

In some embodiments, the formulations can comprise one or more tonicity agents. For example, the formulations can comprise one or more of the above tonicity agents in the above concentrations. In certain specific embodiments, the formulations can comprise trehalose and arginine (e.g., arginine-HCl), wherein each of the trehalose and arginine concentrations is 50-300 mM (e.g., about 50-250 mM, about 50-200 mM, about 50-150 mM, about 100-150 mM, about 100-140 mM, or about 100-130 mM).

In some embodiments, the osmolality of the formulations ranges from about 200 mOsm/kg to about 500 mOsm/kg, about 250 mOsm/kg to about 450 mOsm/kg, about 200 mOsm/kg to about 350 mOsm/kg, about 230 mOsm/kg to about 330 mOsm/kg, about 250 mOsm/kg to about 300 mOsm/kg, about 260 mOsm/kg to about 280 mOsm/kg, about 350 mOsm/kg to about 450 mOsm/kg, about 380 mOsm/kg to about 420 mOsm/kg, e.g., about 270 mOsm/kg or about 450 mOsm/kg. In some embodiments, the formulation is substantially isotonic, i.e., having substantially the same osmotic pressure as blood in a mammal (e.g., a human). Osmolality can be measured by any means known to those of skill in the art, such as using vapor pressure or ice-freezing type osmometers. The osmolality of the formulations described herein can, for instance, be regulated by the one or more tonicity agents described herein.

### Amino Acids

The formulations can optionally further comprise an amino acid. In some embodiments, the amino acid is also a tonicity agent. In some embodiments, the amino acid is a stabilizing agent. Examples of amino acids include, but are not limited to, glycine, alanine, aspartic acid, lysine, serine, tyrosine, cysteine, glutamine, methionine, arginine, and proline. In some embodiments, the amino acid is in its hydrochloride salt form In some embodiments, the amino acid is an L-enantiomer. In some embodiments, the amino acid is a D-enantiomer. In some embodiments, the amino acid is a mixture of L- and D-enantiomers.

In some embodiments, the amino acid is present in the formulations in an amount of 10-500 mM (e.g., about 10-450 mM, about 10-400 mM, about 10-350 mM, about 10-300 mM, about 10-250 mM, about 10-200 mM, about 10-150 mM, about 10-100 mM, about 10-50 mM, about 50-500 mM, about 50-450 mM, about 50-400 mM, about 50-350 mM, about 50-300 mM, about 50-250 mM, about 50-200 mM, about 50-150 mM, about 50-100 mM, about 100-500 mM, about 100-450 mM, about 100-400 mM, about 100-350 mM, about 100-300 mM, about 100-250 mM, about 100-200 mM, about 100-150 mM, about 150-500 mM, about 150-450 mM, about 150-400 mM, about 150-400 mM, about 150-350 mM, about 150-300 mM, about 150-250 mM, about 150-200 mM, about 200-500 mM, about 200-450 mM, about 200-400 mM, about 200-350 mM, about 200-300 mM, about 200-250 mM, about 250-500 mM, about 250-450 mM, about 250-400 mM, about 250-350 mM, about 250-300 mM, about 300-500 mM, about 300-450 mM, about 300-400 mM, about 300-350 mM, about 350-500 mM, about 350-450 mM, about 350-400 mM, about 400-500 mM, about 400-450 mM, or about 450-500 mM).

An exemplary amino acid is arginine (e.g., arginine-HCl). In some embodiments, arginine has a concentration of about 10-200 mM (e.g., about 10-50 mM, about 50-100 mM, about 100-110 mM, about 110-120 mM, about 120-130 mM, about 130-140 mM, about 140-150 mM, about 150-160 mM, about 160-170 mM, about 170-180 mM, about 180-190 mM, or about 190-200 mM). In some embodiments, the formulation described herein includes about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, or about 200 mM of arginine.

### Other Excipients

Furthermore, the formulations can comprise other excipients including, but not limited to, water for injection, diluents, solubilizing agents, soothing agents, additional buffers, inorganic or organic salts, antioxidants, or the like. In some embodiments, however, the formulations have no other excipients, except those described above. Other pharmaceutically acceptable carriers, excipients, or stabilizers, such as those described in Remington's Pharmaceutical Sciences 16th edition, Osol, A. Ed. (1980) may be included in the formulation provided that they do not adversely affect the desired characteristics of the formulation. In a particular embodiment, the formulation is substantially free of preservatives, although, in alternative embodiments, preservatives can be added as necessary. For example, cryoprotectants or lyoprotectants can be included in lyophilized formulations.

In some embodiments, the formulation can further comprise a metal chelator and/or an anti-oxidant, as well as other pharmaceutically acceptable excipients. Suitable metal chelators include, for example, methylamine, ethylenediamine, desferoxamine, trientine, histidine, malate, phosphonate compounds, e.g., etidronic acid, ethylenediaminetetraacetic acid (EDTA), ethyleneglycoltetraacetic acid (EGTA), and the like. Suitable anti-oxidants include, for example, citric acid, uric acid, ascorbic acid, lipoic acid, glutathione, tocopherol, carotene, lycopene, cysteine and the like.

### Exemplary Formulations

In one aspect, the disclosure provides a stable aqueous pharmaceutical formulation comprising an anti-PD-L1/anti-4-1BB bispecific antibody or antigen-binding fragment thereof, and a buffer, wherein the formulation has a pH of about 5.0 to about 6.5.

In some embodiments, the stable aqueous pharmaceutical formulation comprises, consist of, or consists essentially of
a) an anti-PD-L1/anti-4-1BB bispecific antibody or antigen-binding fragment thereof with a concentration of at least 10 mg/ml;
b) a stabilizing agent with a concentration of about 10 mM to about 500 mM;
c) a surfactant with a concentration of about 0.01 mg/ml-about 10 mg/ml; and
d) a buffer system, wherein the formulation has a pH of about 5.0 to about 6.5.

In some embodiments, the stable aqueous pharmaceutical formulation comprises, consist of, or consists essentially of
a) an anti-PD-L1/anti-4-1BB bispecific antibody or antigen-binding fragment thereof with a concentration of about 10 mg/ml to about 100 mg/ml;
b) a tonicity agent with a concentration of about 10 mM to about 500 mM;
c) a surfactant with a concentration of about 0.01 mg/ml-about 10 mg/ml; and
d) a buffer system, wherein the formulation has a pH of about 5.0 to about 6.5.

In some embodiments, the stable aqueous pharmaceutical formulation comprises, consist of, or consists essentially of
e) an anti-PD-L1/anti-4-1BB bispecific antibody or antigen-binding fragment thereof with a concentration of about 10 mg/ml to about 100 mg/ml;
f) an amino acid with a concentration of about 10 mM to about 500 mM;
g) a surfactant with a concentration of about 0.01 mg/ml-about 10 mg/ml; and
h) a buffer system, wherein the formulation has a pH of about 5.0 to about 6.5.

In some embodiments, the stable aqueous pharmaceutical formulation comprises, consist of, or consists essentially of
a) an anti-PD-L1/anti-4-1BB bispecific antibody or antigen-binding fragment thereof with a concentration of about 10 mg/ml to about 100 mg/ml;
b) arginine (e.g., arginine-HC1) with a concentration of about 10 mM to about 500 mM;
c) acetic acid/sodium acetate buffer, wherein the formulation has a pH of about 5.0 to about 6.5.

In some embodiments, the stable aqueous pharmaceutical formulation comprises, consist of, or consists essentially of
a) an anti-PD-L1/anti-4-1BB bispecific antibody or antigen-binding fragment thereof with a concentration of about 10 mg/ml to about 100 mg/ml;
b) arginine (e.g., arginine-HCl) with a concentration of about 10 mM to about 500 mM;
c) trehalose with a concentration of about 10 mM to about 500 mM;
d) acetic acid/sodium acetate buffer, wherein the formulation has a pH of about 5.0 to about 6.5.

In some embodiments, the stable aqueous pharmaceutical formulation comprises, consist of, or consists essentially of
a) an anti-PD-L1/anti-4-1BB bispecific antibody or antigen-binding fragment thereof with a concentration of about 10 mg/ml to about 100 mg/ml;
b) a surfactant with a concentration of about 0.01 mg/ml-about 10 mg/ml;
c) acetic acid/sodium acetate buffer, wherein the formulation has a pH of about 5.0 to about 6.5.

In some embodiments, the stable aqueous pharmaceutical formulation comprises, consist of, or consists essentially of
a) an anti-PD-L1/anti-4-1BB bispecific antibody or antigen-binding fragment thereof with a concentration of about 10 mg/ml to about 100 mg/ml;
b) arginine (e.g., arginine-HCl) with a concentration of about 10 mM to about 500 mM;
c) a surfactant with a concentration of about 0.01 mg/ml-about 10 mg/ml (e.g., about 0.2 mg/ml);
d) acetic acid/sodium acetate buffer, wherein the formulation has a pH of about 5.0 to about 6.5.

In some embodiments, the stable aqueous pharmaceutical formulation comprises, consist of, or consists essentially of
a) an anti-PD-L1/anti-4-1BB bispecific antibody or antigen-binding fragment thereof with a concentration of about 10 mg/ml to about 100 mg/ml;
b) arginine (e.g., arginine-HC1) with a concentration of about 10 mM to about 200 mM;
c) polysorbate 80 with a concentration of about 0.1 mg/ml-about 0.5 mg/ml (e.g., about 0.2 mg/ml);
d) acetic acid/sodium acetate buffer at a concentration of about 10-100 mM, wherein the formulation has a pH of about 5.5 to about 6.0.

In some embodiments, the stable aqueous pharmaceutical formulation comprises, consist of, or consists essentially of
a) an anti-PD-L1/anti-4-1BB bispecific antibody or antigen-binding fragment thereof with a concentration of about 10 mg/ml to about 100 mg/ml;
b) arginine (e.g., arginine-HCl) with a concentration of about 10 mM to about 200 mM;
c) trehalose with a concentration of about 10 mM to about 300 mM;
d) polysorbate 80 with a concentration of about 0.1 mg/ml-about 0.3 mg/ml (e.g., about 0.2 mg/ml);
e) acetic acid/sodium acetate buffer at a concentration of about 10-100 mM, wherein the formulation has a pH of about 5.5 to about 6.0.

In some embodiments, the stable aqueous pharmaceutical formulation comprises, consist of, or consists essentially of
a) an anti-PD-L1/anti-4-1BB bispecific antibody or antigen-binding fragment thereof with a concentration of about 10 mg/ml to about 100 mg/ml;
b) arginine (e.g., arginine-HCl) with a concentration of about 140 mM;
c) polysorbate 80 with a concentration of about 0.2 mg/ml;
d) acetic acid/sodium acetate buffer at a concentration of about 10 mM, wherein the formulation has a pH of about 5.5 to about 6.0 (e.g., 5.6).

In some embodiments, the stable aqueous pharmaceutical formulation comprises, consist of, or consists essentially of
a) an anti-PD-L1/anti-4-1BB bispecific antibody or antigen-binding fragment thereof with a concentration of about 10 mg/ml to about 100 mg/ml;
b) arginine (e.g., arginine-HCl) with a concentration of about 100 mM;
c) trehalose with a concentration of about 130 mM;
d) polysorbate 80 with a concentration of about 0.2 mg/ml;
e) acetic acid/sodium acetate buffer at a concentration of about 15 mM, wherein the formulation has a pH of about 5.5 to about 6.0 (e.g., 5.8).

In some embodiments, the formulation is substantially free from any sugar or any sugar except trehalose. In some embodiments, the formulation is substantially free from any other tonicity agent. In some embodiments, the formulation is substantially free from any other buffering agent. In some embodiments, the formulation is substantially free from any amino acid or any amino acid except arginine.

### Stability

The formulation described herein is stable upon storage. The formulation can be refrigerated (e.g., 2-8° C.) or frozen (e.g., at −20° C., −70° C., or −80° C.) for storage. Contemplated formulations are stable at about 4° C., about 25° C., about 40° C. or at room temperature for at least about 1, 2, 3, or 4 weeks or more; or 1, 2, 3, 4, 5, 6, 7, 8, 9, 10, 11 or 12 months or more, and typically at least about 12, 18, or 24 months, or more. In some embodiments, they are stable at about 25° C. for at least about 6 months or more.

Stability can be tested by evaluating physical stability, chemical stability, and/or biological activity of the antibody or antigen binding fragment thereof in the formulation around the time of formulation as well as following storage at the noted temperatures. Physical and/or chemical stability of a liquid formulation can be evaluated qualitatively and/or quantitatively in a variety of different ways (see, e.g., Analytical Techniques for Biopharmaceutical Development, Rodriguez-Diaz et al. eds. Informa Healthcare (2005)), including evaluation of aggregate formation (for example using size exclusion (or gel filtration) chromatography (SEC), size exclusion high performance liquid chromatography (SEC-HPLC), reverse phased chromatography-ultra performance liquid chromatography (RPC-UPLC), matrix-assisted laser desorption-ionization time-of-flight mass spectrometry (MALDI-TOF MS), analytical ultracentrifugation, light scattering (photon correlation spectroscopy, dynamic light scattering (DLS), multi-angle laser light scattering (MALLS)), flow-based microscopic imaging, electronic impedance (coulter) counting, light obscuration or other liquid particle counting system, by measuring turbidity, by observing appearance of the test samples, density gradient centrifugation and/or by visual inspection); by assessing charge heterogeneity using cation exchange chromatography (see also Vlasak and Ionescu, Curr. Pharm Biotechnol. 9:468-481 (2008) and Harris et al. J. Chromatogr. B Biomed. Sci. Appl. 752:233-245 (2001)), ion exchange high performance liquid chromatography (IEX-HPLC), isoelectric focusing (IEF), e.g. capillary technique (cIEF), or capillary zone electrophoresis; amino-terminal or carboxy terminal sequence analysis; mass spectrometric analysis; SDS-PAGE or SEC analysis to compare fragmented, intact and multimeric (i.e., dimeric, trimeric, etc.) antibody; peptide map (for example tryptic or LYS- and the like); evaluating biological activity or antigen binding function of the antibody; and the like. Biological activity or antigen binding function, e.g., binding of the anti-PD-L1/anti-4-1BB bispecific antibodies to PD-1, PD-L1, or inhibition of the binding between PD-1 and PD-L1; binding of the anti-PD-L1/anti-4-1BB bispecific antibodies to 4-1BB, 4-1BBL, or inhibition of the binding between 4-1BB and 4-1BBL, can be evaluated using various techniques available to the skilled practitioner.

Stability can be measured at a selected temperature for a selected time period. In one aspect, a formulation is stable at about 40° C. for at least about 1, 2, 3, or 4 weeks, at least about 2 months, at least about 3 months, at least about 6 months, at least about 9 months, at least about 12 months, or at least about 18 months. During this period, the formulation is substantially free from any precipitation, and/or the decrease of the protein concentration is less than 0.1, 0.2, 0.3, 0.4, 0.5, 0.6, 0.7, 0.8, 0.9, 1, 2, 3, 4, or 5%. In some embodiments, the decrease of the protein concentration is less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2%, or less than about 1% at 40 after 4 weeks. In some embodiments, the protein concentration is decreased no more than 0.1, 0.2, 0.3, 0.4, 0.5, 0.6, 0.7, 0.8, 0.9, 1, 2, 3, 4, or 5 mg/ml. In some embodiments, the change of the protein purity (e.g., as measured by SEC-HPLC, RPC-UPLC, and/or CE-SDS methods) is less than 0.1, 0.2, 0.3, 0.4, 0.5, 0.6, 0.7, 0.8, 0.9, 1, 1.5, 2, 2.5, 3, 3.5, 4, 4.5, or 5%. In some embodiments, the main peak ratio of charge isomers (e.g., as measured by IEX-HPLC) is less than 15%, 14%, 13%, 12%, 11%, 10%, 9%, 8%, 7%, 6%, 5%, 4%, 3%, 2%, or 1%.

In one aspect, a formulation is stable at about 25° C. for at least 1 month, at least 2 months, at least about 3 months, at least about 6 months, at least about 9 months, at least about 12 months, or at least about 18 months. During this period, the formulation is substantially free from any precipitation, and/or the decrease of the protein concentration is less than 0.1, 0.2, 0.3, 0.4, 0.5, 0.6, 0.7, 0.8, 0.9, 1, 2, 3, 4, or 5%. In some embodiments, the decrease of the protein concentration is less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2%, or less than about 1% at 25 after 6 months. In some embodiments, the protein concentration is decreased no more than 0.1, 0.2, 0.3, 0.4, 0.5, 0.6, 0.7, 0.8, 0.9, 1, 2, 3, 4, or 5 mg/ml. In some embodiments, the change of the protein purity (e.g., as measured by SEC-HPLC, RPC-UPLC, and/or CE-SDS methods) is less than 0.1, 0.2, 0.3, 0.4, 0.5, 0.6, 0.7, 0.8, 0.9, 1, 1.5, 2, 2.5, 3, 3.5, 4, 4.5, or 5%. In some embodiments, the main peak ratio of charge isomers (e.g., as measured by IEX-HPLC) is less than 20%, 19%, 18%, 17%, 16%, 15%, 14%, 13%, 12%, 11%, 10%, 9%, 8%, 7%, 6%, 5%, 4%, 3%, 2%, or 1%.

Instability may involve any one or more of aggregation (e.g., non-covalent soluble aggregation (caused by hydrophobic or charge interactions), covalent soluble aggregation (e.g., disulfide bond rearrangement/scrambling), insoluble aggregation (cause by denaturing of the protein at the liquid/air and liquid/solid interfaces)), deamidation (e.g. Asn deamidation), oxidation (e.g. Met oxidation), isomerization (e.g. Asp isomeriation), denaturation, clipping/hydrolysis/fragmentation (e.g. hinge region fragmentation), succinimide formation, N-terminal extension, C-terminal processing, glycosylation differences, and the like.

In some embodiments, the formulation after storage (e.g., about 40±2° C. for about 4 weeks or about 25±2° C. for 6 months) is colorless and substantially free from any visible foreign matter. In some embodiments, the formulation after storage (e.g., about 40±2° C. for about 4 weeks or about 25±2° C. for 6 months) is substantially free from any precipitation.

In some embodiments, the decrease of the protein concentration after storage (e.g., about 40±2° C. for about 4 weeks or about 25±2° C. for 6 months) is less than 0.1, 0.2, 0.3, 0.4, 0.5, 0.6, 0.7, 0.8, 0.9, 1, 2, 3, 4, 5, 6, 7, 8, 9 or 10%. In some embodiments, the change of the protein purity (e.g., as measured by SEC-HPLC, RPC-UPLC, and/or CE-SDS methods) is less than 0.1, 0.2, 0.3, 0.4, 0.5, 0.6, 0.7, 0.8, 0.9, 1, 2, 3, 4, 5, 6, 7, 8, 9, or 10%.

In some embodiments, the decrease of the protein concentration after 1, 2, 3, 4, 5, 6, 7, 8, 9, or 10 freeze-thaw cycles (e.g., about −70 to about −80° C. for at least 1, 2, 3, 4, 5, or 6 hours followed by 25° C. as one cycle) is less than 0.1, 0.2, 0.3, 0.4, 0.5, 0.6, 0.7, 0.8, 0.9, 1, 2, 3, 4, or 5%. In some embodiments, the change of the protein purity (e.g., as measured by SEC-HPLC, RPC-UPLC, and/or CE-SDS methods) is less than 0.1, 0.2, 0.3, 0.4, 0.5, 0.6, 0.7, 0.8, 0.9, 1, 2, 3, 4, 5, 6, 7, 8, 9, or 10%.

In some embodiments, the decrease of the protein concentration after at least 1 day, 2 days, 3 days, 4 days, or 5 days of agitation (e.g., about 100 rpm, about 150 rpm, about 200 rpm, about 210 rpm, about 220 rpm, about 230 rpm, about 240 rpm, about 250 rpm, about 260 rpm, about 270 rpm, about 280 rpm, about 290 rpm, about 300 rpm, about 350 rpm, about 400 rpm, about 450 rpm, or about 500 rpm) is less than 0.1, 0.2, 0.3, 0.4, 0.5, 0.6, 0.7, 0.8, 0.9, 1, 2, 3, 4, or 5%. In some embodiments, the change of the protein purity (e.g., as measured by SEC-HPLC, RPC-UPLC, and/or CE-SDS methods) is less than 0.1, 0.2, 0.3, 0.4, 0.5, 0.6, 0.7, 0.8, 0.9, 1, 2, 3, 4, 5, 6, 7, 8, 9, or 10%.

In addition, the formulation can be sterile, and this can be achieved according to the procedures known to the skilled person for generating sterile pharmaceutical formulations suitable for administration to subjects, prior to, or following, preparation of the formulation.

### Anti-PD-L1/Anti-4-1BB Bispecific Antibodies and Antigen-Binding Fragments

One aspect of the present invention relates to a bispecific antibody comprising a first antigen-binding functional region that specifically binds to PD-L1 and a second antigen-binding functional region that specifically binds to 4-1BB, wherein the first antigen-binding functional region that specifically binds PD-L1 comprises:
(A) a heavy chain variable region, comprising
   (a) HCDR1, which comprises the amino acid sequence set forth in SEQ ID NO:15,
   (b) HCDR2, which comprises the amino acid sequence set forth in SEQ ID NO:16, and
   (c) HCDR3, which comprises the amino acid sequence set forth in SEQ ID NO:17; and
(B) a light chain variable region, comprising
   (a) LCDR1, which comprises the amino acid sequence set forth in SEQ ID NO:18,
   (b) LCDR2, which comprises the amino acid sequence set forth in SEQ ID NO:19, and
   (c) LCDR3, which comprises the amino acid sequence set forth in SEQ ID NO:20.

In some embodiments, the second antigen-binding functional region of the bispecific antibody that specifically binds to 4-1BB comprises
(A) a heavy chain variable region, comprising
   (a) HCDR1, which comprises the amino acid sequence set forth in SEQ ID NO:21,
   (b) HCDR2, which comprises the amino acid sequence set forth in SEQ ID NO:22, and
   (c) HCDR3, which comprises the amino acid sequence set forth in SEQ ID NO:23; and
(B) a light chain variable region, comprising
   (a) LCDR1, which comprises the amino acid sequence set forth in SEQ ID NO:24,
   (b) LCDR2, which comprises the amino acid sequence set forth in SEQ ID NO:25, and
   (c) LCDR3, which comprises the amino acid sequence set forth in SEQ ID NO:26.

In some embodiments, the first antigen-binding functional region of the bispecific antibody that specifically binds to PD-L1 comprises
(A) a heavy chain variable region, comprising
   (a) HCDR1, which comprises the amino acid sequence set forth in SEQ ID NO:15,
   (b) HCDR2, which comprises the amino acid sequence set forth in SEQ ID NO:16, and
   (c) HCDR3, which comprises the amino acid sequence set forth in SEQ ID NO:17; and
(B) a light chain variable region, comprising
   (a) LCDR1, which comprises the amino acid sequence set forth in SEQ ID NO:18,
   (b) LCDR2, which comprises the amino acid sequence set forth in SEQ ID NO:19, and
   (c) LCDR3, which comprises the amino acid sequence set forth in SEQ ID NO:20; and
the second antigen-binding functional region that specifically binds to 4-1BB comprises
(A) a heavy chain variable region, comprising
   (a) HCDR1, which comprises the amino acid sequence set forth in SEQ ID NO:21,
   (b) HCDR2, which comprises the amino acid sequence set forth in SEQ ID NO:22, and
   (c) HCDR3, which comprises the amino acid sequence set forth in SEQ ID NO:23; and
(B) a light chain variable region, comprising
   (a) LCDRI, which comprises the amino acid sequence set forth in SEQ ID NO:24,
   (b) LCDR2, which comprises the amino acid sequence set forth in SEQ ID NO:25, and
   (c) LCDR3, which comprises the amino acid sequence set forth in SEQ ID NO:26.

The six CDRs of the first antigen-binding functional region that specifically binds to PD-L1 as set forth in SEQ ID NOs:15-20, and the six CDRs of the second antigen-binding functional region that specifically binds to 4-1BB as set forth in SEQ ID NOs:21-26 are the CDRs of the heavy and light chain variable regions of the monoclonal antibodies obtained by the inventors using hybridoma technology with human PD-L1 and 4-1BB as the antigens. Said monoclonal antibodies are different from the PD-L1 antibodies and 4-1BB antibodies known in the prior art and have higher biological activities, such as higher binding specificity, anti-tumor activity and so on.

The six CDR sequences of the first antigen-binding functional region that specifically binds to PD-L1 may have at least 70%, such as at least 75%, 80%, 85%, 90%, 95% or higher identity to the sequences set forth in SEQ ID NOs:15-20, respectively, and retain the biological activities of the corresponding parent sequences; or may contain one or more, such as 1, 2, 3 or more amino acid deletions, substitutions and/or additions compared to the sequences set forth in SEQ ID NOs:15-20, respectively, and retain the biological activities of the corresponding parent sequences.

The six CDR sequences of the second antigen-binding functional region that specifically binds to 4-1BB may have at least 70%, such as at least 75%, 80%, 85%, 90%, 95% or higher identity to the sequences set forth in SEQ ID NOs:21-26, respectively, and retain the biological activities of the corresponding parent sequences; or may contain one or more, such as 1, 2, 3 or more amino acid deletions, substitutions and/or additions compared to the sequences set forth in SEQ ID NOs:21-26, respectively, and retain the biological activities of the corresponding parent sequences.

In some embodiments, the antigen-binding functional region that specifically binds to PD-L1 comprises a heavy chain variable region (VH) comprising HCDR1, HCDR2, and HCDR3 that are identical to HCDR1, HCDR2, and HCDR3 of SEQ ID NO:6; and a light chain variable region (VL) comprising LCDR1, LCDR2, and LCDR3 that are identical to LCDR1, LCDR2, and LCDR3 of SEQ ID NO:2.

In some embodiments, the antigen-binding functional region that specifically binds to 4-1BB comprises a heavy chain variable region (VH) comprising HCDR1, HCDR2, and HCDR3 that are identical to HCDR1, HCDR2, and HCDR3 of SEQ ID NO:12; and a light chain variable region (VL) comprising LCDR1, LCDR2, and LCDR3 that are identical to LCDR1, LCDR2, and LCDR3 of SEQ ID NO:10.

In some embodiments, the antibody or the antigen-binding fragment described herein can contain a light chain variable region containing one, two, or three of the CDRs as described herein with zero, one or two amino acid insertions, deletions, or substitutions. In some embodiments, the antibody or the antigen-binding fragment described herein can contain a heavy chain variable region containing one, two, or three of the CDRs as described herein with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat numbering scheme. In some embodiments, the CDR is determined based on Chothia numbering scheme.

In some embodiments, the first antigen-binding functional region of the bispecific antibody that specifically binds to PD-L1 comprises a heavy chain variable region and a light chain variable region, wherein the heavy chain variable region comprises the amino acid sequence set forth in SEQ ID NO:6; or has at least 70%, such as at least 75%, 80%, 85%, 90%, 95%, 96%, 97%, 98%, 99% or higher identity to the sequence set forth in SEQ ID NO:6 and retains the biological activities of the corresponding parent sequence; or contains one or more, such as 1, 2, 3, 4, 5, 6, 7, 8, 9, 10, 15, 20 or more amino acid deletions, substitutions and/or additions compared to the sequence set forth in SEQ ID NO:6 and retains the biological activities of the corresponding parent sequence; and the light chain variable region comprises the amino acid sequence set forth in SEQ ID NO:2; or has at least 70%, such as at least 75%, 80%, 85%, 90%, 95%, 96%, 97%, 98%, 99% or higher identity to the sequence set forth in SEQ ID NO:2 and retains the biological activities of the corresponding parent sequence; or contains one or more, such as 1, 2, 3, 4, 5, 6, 7, 8, 9, 10, 15, 20 or more amino acid deletions, substitutions and/or additions compared to the sequence set forth in SEQ ID NO:2 and retains the biological activities of the corresponding parent sequence.

In some embodiments, the second antigen-binding functional region of the bispecific antibody that specifically binds to 4-1BB comprises a heavy chain variable region and a light chain variable region, wherein the heavy chain variable region comprises the amino acid sequence set forth in SEQ ID NO:12; or has at least 70%, such as at least 75%, 80%, 85%, 90%, 95%, 96%, 97%, 98%, 99% or higher identity to the sequence set forth in SEQ ID NO: 12 and retains the biological activities of the corresponding parent sequence; or contains one or more, such as 1, 2, 3, 4, 5, 6, 7, 8, 9, 10, 15, 20 or more amino acid deletions, substitutions and/or additions compared to the sequence set forth in SEQ ID NO:12 and retains the biological activities of the corresponding parent sequence; and the light chain variable region comprises the amino acid sequence set forth in SEQ ID NO:10; or has at least 70%, such as at least 75%, 80%, 85%, 90%, 95%, 96%, 97%, 98%, 99% or higher identity to the sequence set forth in SEQ ID NO:10 and retains the biological activities of the corresponding parent sequence; or contains one or more, such as 1, 2, 3, 4, 5, 6, 7, 8, 9, 10, 15, 20 or more amino acid deletions, substitutions and/or additions compared to the sequence set forth in SEQ ID NO:10 and retains the biological activities of the corresponding parent sequence.

In some embodiments, the first antigen-binding functional region of the bispecific antibody that specifically binds to PD-L1 comprises a heavy chain variable region and a light chain variable region, wherein the heavy chain variable region comprises the amino acid sequence set forth in SEQ ID NO:6, and the light chain variable region comprises the amino acid sequence set forth in SEQ ID NO:2; and the second antigen-binding functional region that specifically binds to 4-1BB comprises a heavy chain variable region and a light chain variable region, wherein the heavy chain variable region comprises the amino acid sequence set forth in SEQ ID NO:12, and the light chain variable region comprises the amino acid sequence set forth in SEQ ID NO:10.

Those skilled in the art would know that the combinations of the amino acid sequences should follow the biological laws, i.e., light and heavy chains or the variable regions, the antigen-binding fragments thereof, such as SEQ ID NO:6 and SEQ ID NO:2, and SEQ ID NO:12 and SEQ ID NO:10, should match each other.

In some embodiments, the first antigen-binding functional region and the second antigen-binding functional region of the bispecific antibody are selected from the group consisting of Fab fragments, scFv fragments, and variable domain fragments Fv.

In some embodiments, the first antigen-binding functional region and the second antigen-binding functional region of the bispecific antibody are both Fab fragments.

In some embodiments, the Fab fragment of the bispecific antibody comprises a different first heavy chain variable region and a different second heavy chain variable region, as well as a different first light chain variable region and a different second light chain variable region.

In some embodiments, one of the first antigen-binding functional region and the second antigen-binding functional region of the bispecific antibody is a Fab fragment, and the other is a scFv.

In some embodiments, the bispecific antibody comprises a first Fc chain and a second Fc chain, and a first antigen-binding functional region capable of specifically binding to PD-L1 and a second antigen-binding functional region capable of specifically binding to 4-1BB,
wherein the first Fc chain and the second Fc chain are both immunoglobulin G Fc fragments containing amino acid substitutions, and the first Fc chain and the second Fc chain together constitute a heterodimer that can bind to the Fc receptor;
wherein the first Fc chain and the second Fc chain are linked to the first antigen-binding functional region and the second antigen-binding functional region, respectively, via a covalent bond or a linker; and
wherein either the first Fc chain or the second Fc chain comprises amino acid substitutions at positions 366 and 399, and the other comprises amino acid substitutions at positions 351, 407 and 409, wherein the amino acid positions are numbered according to the Kabat EU index numbering system.

In some embodiments, the amino acid substitutions of the first Fc chain and the second Fc chain of the bispecific antibody are as follows,
a) L351G, L351Y, L351V, L351P, L351D, L351E, L351K or L351W;
b) T366L, T366P, T366W or T366V;
c) D399C, D399N, D399I, D399G, D399R, D399T or D399A;
d) Y407L, Y407A, Y407P, Y407F, Y407T or Y407H; and
e) K409C, K409P, K409S, K409F, K409V, K409Q or K409R.

In some embodiments, the amino acid substitutions of the bispecific antibody include:
a) substitutions T366L and D399R on either the first Fc chain or the second Fc chain, and substitutions L351E, Y407L and K409V on the other;
b) substitutions T366L and D399C on either the first Fc chain or the second Fc chain, and substitutions L351G, Y407L and K409C on the other;
c) substitutions T366L and D399C on either the first Fc chain or the second Fc chain, and substitutions L351Y, Y407A and K409P on the other;
d) substitutions T366P and D399N on either the first Fc chain or the second Fc chain, and substitutions L351V, Y407P and K409S on the other;
e) substitutions T366W and D399G on either the first Fc chain or the second Fc chain, and substitutions L351D, Y407P and K409S on the other;
f) substitutions T366P and D399I on either the first Fc chain or the second Fc chai, and substitutions L351P, Y407F and K409F on the other;
g) substitutions T366V and D399T on either the first Fc chain or the second Fc chain, and substitutions L351K, Y407T and K409Q on the other;
h) substitutions T366L and D399A on either the first Fc chain or the second Fc chain, and substitutions L351W, Y407H and K409R on the other.

In some embodiments, the amino acid substitutions of the bispecific antibody include:
a) substitutions T366L and K409V on either the first Fc chain or the second Fc chain, and substitutions L351E, Y407L and D399R on the other;
b) substitutions T366L and K409C on either the first Fc chain or the second Fc chain, and substitutions L351G, Y407L and D399C on the other;
c) substitutions T366L and K409P on either the first Fc chain or the second Fc chain, and substitutions L351Y, Y407A and D399C on the other;
d) substitutions T366P and K409S on either the first Fc chain or the second Fc chain, and substitutions L351V, Y407P and D399N on the other;
e) substitutions T366W and K409S on either the first Fc chain or the second Fc chain, and substitutions L351D, Y407P and D399G on the other;
f) substitutions T366P and K409F on either the first Fc chain or the second Fc chain, and substitutions L351P, Y407F and D399I on the other;
g) substitutions T366V and K409Q on either the first Fc chain or the second Fc chain, and substitutions L351K, Y407T and D399T on the other;
h) substitutions T366L and K409R on either the first Fc chain or the second Fc chain, and substitutions L351W, Y407H and D399A on the other.

In some embodiments, the substitutions on either the first Fc chain or the second Fc chain of the bispecific antibody are T366L and D399R, and the substitutions on the other are L351E, Y407L and K409V.

In some embodiments, the weight ratios of the homodimers formed by the first Fc chain and the first antigen-binding functional region covalently linked thereto and by the second Fc chain and the second antigen-binding functional region covalently linked thereto of said bispecific antibody are less than 50%, such as less than 45%, 40%, 35%, 30%, 25%, 20% or less of the total amount of all the polypeptide chains, in a reducing agent-containing solution in which no other polypeptides present except for said Fc chains and the antigen-binding functional regions hereinabove.

In some embodiments, the bispecific antibody comprises a first heavy chain/first light chain pair that specifically binds to PD-L1, wherein the first heavy chain has an heavy chain variable region comprising the amino acid sequence set forth in SEQ ID NO:6, and a heavy chain constant region comprising the amino acid sequence set forth in SEQ ID NO:8; the first light chain has a light chain variable region comprising the amino acid sequence set forth in SEQ ID NO:2, and a light chain constant region comprising the amino acid sequence set forth in SEQ ID NO:4.

In some embodiments, the bispecific antibody comprises a second heavy chain/second light chain pair that specifically binds to 4-1BB, wherein the second heavy chain has an heavy chain variable region comprising the amino acid sequence set forth in SEQ ID NO:12, and a heavy chain constant region comprising the amino acid sequence set forth in SEQ ID NO:14; the second light chain has a light chain variable region comprising the amino acid sequence set forth in SEQ ID NO:10, and a light chain constant region comprising the amino acid sequence set forth in SEQ ID NO:4.

A second aspect of the present invention relates to an isolated polynucleotide encoding the bispecific antibody in a heterodimeric form as described above.

In some embodiments, the nucleotide sequences encoding the amino acid sequences of the first antigen-binding functional region comprise the nucleotide sequences set forth in SEQ ID NOs:1 and 5.

In some embodiments, the nucleotide sequences encoding the amino acid sequences of the second antigen-binding functional region comprise the nucleotide sequences set forth in SEQ ID NOs:11 and 9.

In some embodiments, the nucleotide sequence encoding the amino acid sequences of the first light chain and the second light chain both comprises the nucleotide sequence set forth in SEQ ID NO:3.

In some embodiments, the nucleotide sequence encoding the amino acid sequence of either the first heavy chain or the second heavy chain comprises the nucleotide sequence set forth in SEQ ID NO:7, and the nucleotide sequence encoding the amino acid sequence of the other comprises the nucleotide sequence set forth in SEQ ID NO:13.

A third aspect of the present invention relates to a recombinant expression vector comprising the isolated polynucleotide as described above.

In some embodiments, the expression vector is the plasmid vector XOGC modified from the pCDNA vector.

A fourth aspect of the present invention relates to a host cell comprising the isolated polynucleotide as described above or the recombinant expression vector as described above.

In some embodiments, the host cell is selected from the group consisting of human embryonic kidney cells HEK293, or HEK293T, HEK293E, HEK293F that are modified from HEK293 cells; hamster ovary cells CHO, or CHO-S, CHO-dhfr, CHO/DG44, ExpiCHO that are modified from CHO cells; Escherichia coli, or E. coli BL21, BL21 (DE3), Rosetta, Origami that are modified from E. coli; Yeast, or Pichia, Saccharomyces cerevisiae, Kluyveromyces lactis, Hansenula polymorpha that are modified from Yeast; insect cells, or High5, SF9 cells that are modified from insect cells; plant cells; mammalian mammary gland cells; somatic cells.

A fifth aspect of the present invention relates to a composition comprising the bispecific antibody as described above or the isolated polynucleotide as described above or the recombinant expression vector as described above or the host cell as described above, and a pharmaceutically acceptable carrier.

A sixth aspect of the present invention relates to a method of producing the bispecific antibody as described above, including the steps of.
1) Expressing the isolated polynucleotide as described above or the recombinant expression vector as described above in host cells, respectively;
2) Reducing the proteins respectively expressed in the host cells; and
3) Mixing the reduced proteins, and then oxidizing the mixture.

In some embodiments, the host cell is selected from the group consisting of human embryonic kidney cells HEK293, or HEK293T, HEK293E and HEK293F that are modified from HEK293 cells; hamster ovary cells CHO, or CHO-S, CHO-dhfr-, CHO/DG44, ExpiCHO that are modified from CHO cells; Escherichia coli, or E. coli BL21, BL21 (DE3), Rosetta, Origami that are modified from E. coli; Yeast, or Pichia, Saccharomyces cerevisiae, Kluyveromyces lactis, Hansenula polymorpha that are modified from Yeast; insect cells, or High5, SF9 cells that are modified from insect cells; plant cells; mammalian mammary gland cells, somatic cells.

In some embodiments, the reduction step includes 1) performing the reduction reaction in the presence of a reducing agent selected from the group consisting of 2-mercaptoethylamine, dithiothreitol, tris(2-carboxyethyl) phosphine, cysteine or other chemical derivatives thereof, 2) Removing the reducing agent. In some embodiments, the reducing agent is dithiothreitol at a concentration of 0.1 mM or higher, such as 0.1 mM, 0.2 mM, 0.3 mM, 0.4 mM, 0.5 mM or higher, and the reaction is performed at 4° C. for at least 3 hours, such as 3.5 hours, 4 hours, 4.5 hours, 5 hours, 5.5 hours, 6 hours or longer.

In some embodiments, the oxidation step is conducted in the air or in the presence of an oxidizing agent selected from the group consisting of L-dehydroascorbic acid, cystamineor chemical derivatives thereof. In some embodiments, the oxidizing agent is L-dehydroascorbic acid at a concentration of 0.5 mM or higher, such as 0.6 mM, 0.7 mM, 0.8 mM, 0.9 mM, 1.0 mM, 1.2 mM, 1.5 mM or higher, and the reaction is performed at 4° C. for at least 5 hours, such as 5 hours, 6 hours, 7 hours, 8 hours, 9 hours, or longer.

In some embodiments, the method further includes a step of separation and purification. In some embodiments, the separation and purification include cation resin exchange, anion resin exchange, reverse phase chromatography, affinity chromatography, size exclusion chromatography, and combinations thereof.

A seventh aspect of the present invention relates to use of the bispecific antibody as described above and/or the isolated polynucleotide as described above and/or the recombinant expression vector as described above and/or the host cell as described above and/or the composition as described above, in the manufacture of a medicament for preventing and/or treating diseases in a subject.

An eighth aspect of the present invention relates to the bispecific antibody as described above and/or the isolated polynucleotide as described above and/or the recombinant expression vector as described above and/or the host cell as described above and/or the composition as described above, for use as a medicament for preventing and/or treating diseases in a subject.

A ninth aspect of the present invention relates to a method of preventing and/or treating diseases, including administering to a subject in need thereof the bispecific antibody as described above and/or the isolated bispecific antibody as described above and/or the recombinant expression vector as described above and/or the host cell as described above and/or the composition as described above.

In some embodiments, the subject is a mammal, preferably a human subject.

In some embodiments, the disease is selected from the group consisting of the following tumors: leukemia, lymphoma, myeloma, brain tumor, head and neck squamous cell cancer, non-small cell lung cancer, nasopharyngeal cancer, esophageal cancer, gastric cancer, pancreatic cancer, gallbladder cancer, liver cancer, colorectal cancer, breast cancer, ovarian renal cell cancer, melanoma, small cell lung cancer and bone cancer.

In other words, the present disclosure designs a novel anti-PD-L1/anti-4-1BB bispecific antibody in the form of a natural antibody structure-like heterodimer with the characteristics of natural IgGs and no heavy and light chain mismatches, and thus it is a highly stable anti-PD-L1/anti-4-1BB bispecific antibody in a heterodimeric form The bispecific antibody prepared in the disclosure is able to bind to two target molecules, PD-L1 and 4-1BB simultaneously, and to exert better efficacy with fewer side effects than a monotherapeutic agent when applied to treat complicated diseases. Moreover, compared to a combination therapy of multiple medicaments, the bispecific antibody, as a single therapeutic molecule, not only facilitates the use by patients and medical workers, but also simplifies the complex development processes for new drugs.

In some embodiments, the antibody or antigen-binding fragment thereof described herein is described in detail in PCT Application No. PCT/CN2022/070624, which is incorporated herein by reference in its entirety.

### Methods of Treatment

The antibodies or antibody or antigen-binding fragments thereof or the formulation comprising the antibodies or antibody or antigen-binding fragments thereof of the present disclosure can be used for various therapeutic purposes. In one aspect, the disclosure provides methods for treating a cancer in a subject, methods of reducing the rate of the increase of volume of a tumor in a subject over time, methods of reducing the risk of developing a metastasis, or methods ofreducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of an antibody or antigen-binding fragment thereof or a formulation comprising the antibody or antigen-binding fragment thereof disclosed herein to a subject in need thereof (e.g., a subject having, or identified or diagnosed as having, a cancer). In some embodiments, the cancer is selected from the group consisting of the following tumors: leukemia, lymphoma, myeloma, brain tumor, head and neck squamous cell cancer, non-small cell lung cancer, nasopharyngeal cancer, esophageal cancer, gastric cancer, pancreatic cancer, gallbladder cancer, liver cancer, colorectal cancer, breast cancer, ovarian renal cell cancer, melanoma, small cell lung cancer and bone cancer.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the antibody, antigen binding fragment, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody or an antigen binding fragment is an amount sufficient to ameliorate, stop, stabilize, reverse, inhibit, slow and/or delay progression of a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an antibody or antigen binding fragment or a formulation comprising the antibody or antigen-binding fragment thereof may vary, depending on, inter alia, patient history as well as other factors such as the type (and/or dosage) of antibody used.

### Kits

Also provided are kits comprising a formulation as described herein. The kit can further comprise one or more containers comprising pharmaceutically acceptable excipients, and include other materials desirable from a commercial and user standpoint, including filters, needles and syringes. Associated with the kits can be instructions customarily included in commercial packages of therapeutic, prophylactic or diagnostic products, that contain information about, for example, the indications, usage, dosage, manufacture, administration, contra-indications, and/or warnings concerning the use of such therapeutic, prophylactic or diagnostic products. The kit can also be associated with a label that can be any kind of data carrier (e.g., a leaflet, sticker, chip, print or bar code) comprising information. In certain embodiments, the methods of use as discussed herein can be comprised in or on the label. The kit can further comprise a device for administration of the formulation, and particularly a device that contains the formulation, ie., a pre-filled device such as, but not limited to, a pre-filled syringe or a pre-filled autoinjector. The kit can also comprise a container comprising the formulation, i.e., a pre-filled container, such as a pre-filled vial, cartouche, sachet, or ampoule.

The formulation can be included in a container, pack, or dispenser together with instructions for administration. The disclosure also provides methods of manufacturing the antibodies or antigen binding fragments thereof for various uses as described herein.

### EXAMPLES

The invention is further described in the following examples, which do not limit the scope of the invention described in the claims.

### Methods and Materials

In order to develop a formulation for long-term stable storage of the recombinant anti-PD-L1/anti-4-1BB bispecific antibody injection solution as described in PCT Application No. PCT/CN2022/070624 and to ensure the quality of the product within its shelf life (e.g., at least 24 months), experiments were designed to screen formulations including the anti-PD-L1/anti-4-1BB bispecific antibody. Effects of different excipients on the stability of the antibody preparations were evaluated. The materials and methods used in the test are as follows:

TABLE 1
Materials used in formulation studies

| Name | Manufacturer | Catalog No. |
|---|---|---|
| Sodium acetate | Sigma-Aldrich | 71183-250G |
| Acetic acid | Sigma-Aldrich | 33209-1L |
| Sodium hydroxide | Sigma-Aldrich | 6203 |
| Trehalose | Sigma-Aldrich | T9531 |
| Trehalose | Sinopharm | 63012666 |
| L-arginine monohydrochloride | Merck | 1.01544.1000 |
| Polysorbate 80 | Sigma-Aldrich | P1754-25ML |

TABLE 2
Instruments used in formulation studies

| Name | Manufacturer | Model |
|---|---|---|
| Centrifuge | Eppendorf | 5424R/5430R/ST-16R |
| Balance | METTLER TOLEDO | XSE204 |
| Ultrapure water system | Millipore | Milli-Q |
| pH meter | METTLER TOLEDO | S470 |
| Constant temperature incubator | BOXUN | BSP-400 |
| Tangential flow ultrafiltration concentration | Millipore | Labscale ™ TFF |
| system (TFF system) | | |
| UV spectrophotometer | GE | Ultrospec 8000 |
| HPLC | Agilent | 1200, 1260 series |
| HPLC/UPLC | Waters | Acquity H-class |
| Capillary Electrophoresis System | Beckman coulter | CESI 8000 Plus |
| (CE system) | | |
| Automatic osmometer | Gonotec | Osmomat auto |
| Vortex | Scientific Industries | VORTEX-GENIE2 |

TABLE 3
Test samples

| Name | Lot No. | Conc. (mg/mL) |
|---|---|---|
| BH3120 | L31282-210223UF/DF | 50.90 |
| | L31283-2104301UF/DF | 46.16 |

Stability of the test samples in different formulations were tested by the methods described as follows.

### Appearance

The appearance of the test samples was observed according to relevant methods described in Chinese Commission Pharmacopoeia. “Pharmacopoeia of the People's Republic of China.” Pharmacopoeia of the People's Republic of China (2020): 122-129. Specifically, the inspector should have 4.9 or above on both distance and near vision tests (corrected visual acuity of 5.0 or above) and should be free of color blindness. The inspector can adjust the position so that the test sample is located at the photopic distance of the eye (referring to the clear observation distance from the test sample to the human eye, usually 25 cm). The test samples were placed under the diffused daylight, and observed horizontally against a white background. The inspector then determined whether the color and clarity of the test samples were similar to that of water in the same container, and whether there was obvious turbidity or precipitation.

### Protein Concentration

The UV-Vis absorption spectrum of protein is the absorption spectrum produced by the electronic energy level transition after some groups in the molecule absorb UV and/or visible light. Protein molecules contain aromatic amino acids with conjugated double bonds, e.g., tyrosine and tryptophan. They have a maximum absorbance at the wavelength of about 280 nm Within a certain range, their absorbance is directly proportional to the protein concentration. Based on the Beer-Lambert law (A=a×b×c, where A is absorbance, a is extinction coefficient, b is detection optical path, and c is protein concentration of the sample), the protein concentration can be calculated.

The protein concentration was determined by a UV-Vis spectrophotometer (GE, Ultrospec™ 8000). Before measurement, the UV-Vis spectrophotometer was turned on and preheated for 30 minutes. The test samples were diluted with water or corresponding buffer solution such that the signal was between 0.200 to 0.800. After zeroing with the buffer solution or water, the appropriately diluted sample was added into the detection cell for measurement.

pH

A pH meter was used for pH measurement. Prior to the measurement, the samples and the calibration buffers were pre-warmed to room temperature. The pH meter was turned on and an appropriate calibration mode was selected. Standard buffer solutions were selected based on an estimate of the approximate pH range of the samples. The electrode was washed with deionized water or ultrapure water, and then dried by wiping the remaining water. Calibration was performed and the pH of the samples were measured after confirmation of a calibration slope between 95-105% with a deviation around 0±30 mV. The electrode was washed with deionized water or ultrapure water, and then dried by wiping the remaining water. Afterwards, the electrode was dipped into the sample and the “Read” button was pressed. The sample was gently shaken until the reading was stable, and then the sample pH was recorded.

### SEC-HPLC

Size exclusion high performance liquid chromatography, or SEC-HPLC, is an important method for antibody quality control. This method can separate molecules based on the molecule size or the difference of the hydrodynamic radius. By SEC-HPLC, three major forms of antibodies can be separated: the high molecular weight form (HMW), the main peak (mainly antibody monomers), and the low molecular weight form (LMW). By SEC-HPLC, the percentage of antibody monomers in the samples can be determined, therefore providing information about the content of soluble aggregates and fragments.

The SEC-HPLC method described herein used XBridge BEH SEC 200 chromatography column. Specifically, 100 mM phosphate buffer (PB)+100 mM NaCl solution (pH about 6.7±0.1) was prepared as the mobile phase buffer for isocratic elution. The injection volume was 10 μl, the flow rate was 0.8 ml/min, the acquisition time was 25 minutes, the column temperature was 25° C., and the detection wavelength was 280 nm. Each sample was diluted to 1 mg/ml with the mobile phase buffer as the test solution for detection.

### IEX-HPLC

Ion exchange high performance liquid chromatography (IEX-HPLC), especially cation exchange high performance liquid chromatography (CEX-HPLC), is commonly used to determine the charge isomers of antibodies. In the assays described herein, peaks eluted from the column with an earlier retention time than the main peak were named as “acidic peaks”, while those eluted from the column with a later retention time than the main peak were marked as “basic Peaks”. The IEX-HPLC method described herein used MAbPac™ SCX-10 chromatography column for detection, mobile phase A: 20 mM MES, 10 mM NaCl, pH 5.6±0.1; mobile phase B: 20 mM MES, 300 mM NaCl, pH 5.6±0.1. The injection volume was 40 μl, the flow rate was 0.6 ml/min, the acquisition time was 62 minutes, the column temperature was 25° C., and the detection wavelength was 280 nm. Each sample was diluted to 1 mg/ml with the mobile phase A buffer as the test solution for detection.

CE-SDS (R/NR)

Capillary electrophoresis-SDS (CE-SDS) is an antibody purity determination method using capillaries as separation channels. In CE-SDS, protein migration is driven by the surface charge caused by SDS binding, which is directly proportional to the molecular weight of the protein. Because all SDS-protein complexes have similar mass-to-charge ratio, electrophoresis separation based on molecular size or hydrodynamic radius can be achieved in the molecular sieve gel matrix of capillary. This method has been widely used to detect the purity of antibodies. Generally, in the non-reduced CE-SDS method, the test sample was mixed with SDS sample buffer and iodoacetamide. After that, the mixture was incubated at 68-72° C. for about 10-15 minutes. After cooling to room temperature, the mixture was centrifuged and the supernatant was used for analysis. The migration of protein was detected by a UV detector and the electrophoretic spectrum was obtained. The purity of antibody can be calculated as the percentage of the peak area of IgG main peak to the sum of all peak areas. In the reduced CE-SDS method, the test sample was mixed with SDS sample buffer and 2-mercaptoethanol. Then the mixture was incubated at 68-72° C. for about 10-15 minutes. After cooling to room temperature, the mixture was centrifuged and the supernatant was used for analysis. The purity of antibody can be calculated as the percentage of the sum of light chains peak areas and heavy chains peak areas to the sum of all peak areas.

The capillary was a fused silica capillary with an inner diameter of 50 μm. The total length was 30.2 cm and the effective length was 20.2 cm. Before electrophoresis, 0.1 mol/L sodium hydroxide, 0.1 mol/L hydrochloric acid, ultrapure water and electrophoresis gel were successively used to equilibrate the capillary column. When the protein concentration was less than 10 mg/ml and the salt concentration was greater than 50 mM, the sample buffer was replaced with water or other low-salt buffer through ultrafiltration to avoid adverse effects caused by the high concentration of salt. 100 μg sample (less than 45 μl) was transferred to a 0.5 ml micro-centrifuge tube, and sample buffer was added to a total volume of 95 μl. Then, 2 μl internal standard (10 kDa), 5 μl iodoacetamide solution (250 mM) or 5 μl 2-mercaptoethanol were added. After complete mixing, the tube was centrifuged at 300 g for 1 minute. After centrifugation, the tube was sealed using parafilm, and then incubated in a 70° C. water bath for 10 minutes, followed by cooling to room temperature for at least 3 minutes. Afterwards, 100 μl sample was transferred into a 200 μl micro sample tube, and all air bubbles were eliminated. The samples were kept in a general-purpose bottle and the cap was closed. The following conditions were used for detection: sample injection: 5 kV, 40 seconds; separation voltage: 15 kV, 40 minutes. The capillary column temperature was kept at 25° C., and the detection wavelength was 214 nm

### RPC-UPLC

Reverse Phased Chromatography-Ultra Performance Liquid Chromatography (RPC-UPLC) can be used to detect samples using MAbPac™ RP column (analytical 4 μm, 2.1×100 mm, PN: 088647, Thermo) and Waters™ ultra-high pressure liquid system (H class plus). Mobile phase A was 0.1% TFA/Water solution; mobile phase B was 0.1% TFA/acetonitrile solution. Injection volume was 5 μL; detection wavelength was 214 nm (UV); column oven temperature was 70° C.; injector temperature is about 2-8° C. Each sample was diluted to 1 mg/ml with water as the test solution for detection.

### Example 1. Preparation and Purification of Anti-PD-L1/Anti-4-1BB Bispecific Antibodies

Same experiments were performed as described in examples of PCT Application No. PCT/CN2022/070624, to prepare and purify BH3120, an anti-PD-L1/anti-4-1BB bispecific antibody, which comprises: an anti-PD-L1 arm including a heavy chain variable region with amino acid sequence set forth in SEQ ID NO:6, a heavy chain constant region with amino acid sequence set forth in SEQ ID NO:8, a light chain variable region with amino acid sequence set forth in SEQ ID NO:2, and a light chain constant region with amino acid sequence set forth in SEQ ID NO:4; and an anti-4-1BB arm including a heavy chain variable region with amino acid sequence set forth in SEQ ID NO:12, a heavy chain constant region with amino acid sequence set forth in SEQ ID NO:14, a light chain variable region with amino acid sequence set forth in SEQ ID NO:10, and a light chain constant region with amino acid sequence set forth in SEQ ID NO:4.

### Example 2. Preparation of Antibody Formulations

According to the experimental results described in examples of PCT Application No. PCT/CN2022/070624, possible components and concentrations in BH3120 formulation were estimated. Factors to be investigated include pH, acetate, arginine-HCL and trehalose. The factors investigated in the experiment and their concentration ranges are shown in the table below.

TABLE 4
Main factors and range to be investigated

| Factor | Range |
|---|---|
| pH | 5.0~6.5 |
| Acetate | 10~100 mM |
| Trehalose | 0~275 mM |
| Arginine-HCL | 0~140 mM |

The corresponding blank formulation buffers (excluding the antibody protein and polysorbate 80) were prepared. The BH3120 protein was centrifuged by ultrafiltration to exchange buffer, and the protein concentration was adjusted to 20 mg/mL, 50 mg/mL, or 80 mg/mL. Polysorbate 80 was added to a final concentration of 0.2 mg/ml. After further filtration and sterilization, the samples were dispensed into vials (about 0.2 mL/vial). Stability of the samples was investigated under the conditions of 40° C. and 25° C. incubation, freeze-thaw cycles (between −70° C. and room temperature) and agitation (250 rpm). The exemplary composition of two formulations are shown in the table below.

TABLE 5
Composition of formulations

| Formulation 1 | | | Formulation 2 | | |
|---|---|---|---|---|---|
| pH | 5.8-6.0 | | pH | 5.6-5.8 | |
| Acetate | 15 | mM | Acetate | 10 | mM |
| Trehalose | 130 | mM | Arg-HCL | 140 | mM |
| Arg-HCL | 100 | mM | Polysorbate 80 | 0.2 | mg/ml |
| Polysorbate 80 | 0.2 | mg/ml | BH3120 protein | 50 | mg/mL |

| | | | | |
|---|---|---|---|---|
| BH3120 protein | 50 | mg/mL | / | / |

### Example 3. Confirmation of Antibody Formulations

The BH3120 protein samples were prepared and tested under different experimental conditions. The protein stability in different formulations were compared. The experiments were performed as shown in the table below.

TABLE 6
Experiments to test BH3120 protein stability in different formulations

| | Conc. | | Condition/Sampling | |
|---|---|---|---|---|
| Sample | (mg/mL) | Buffer | Point | Test Items |
| BH3120 | 20 | Acetate buffer | 40° C. (0/1/2/4 week) | Appearance |
| Formulation 1 | 50 | pH 5.8 | 25° C. (1/2/3/6 month) | Concentration |
| | 80 | | Freeze/thraw (3/5 cycles) | pH |
| BH3120 | 50 | Acetate buffer | Agitate (250 rpm, | SEC-HPLC |
| Formulation 2 | | pH 5.6 | 1/3/5 day) | IEX-HPLC |
| | | | | CE-SDS(R/NR) |
| | | | | RPC-UPLC |

### 40° C. Incubation Test

The protein samples were placed in a constant temperature incubator at 40° C. in the dark for 4 weeks. The changes of each test item were detected by sampling at 0 week, 1 week, 2 weeks, and 4 weeks, respectively. Samples of each preparation at 40° C. for 0 weeks, 1 week, 2 weeks, and 4 weeks were all colorless and clear liquids, with no obvious turbidity or precipitation. Changes in protein concentration, pH, purity and charge isomers are shown in the table below. The results showed that the change of protein concentration within 4 weeks at 40° C. was less than 10%, the decrease of SEC-HPLC purity was less than 3% within 2 weeks at 40° C., and the main peak ratio of charge isomers decreased by less than 10% within 4 weeks at 40° C. Within 4 weeks at 40° C., the purity of non-reduced and reduced CE-SDS decreased by less than 3%.

TABLE 7
Results of formulation confirmation at 40° C.

| | | | | | | | | CE-SDS- | CE-SDS- |
|---|---|---|---|---|---|---|---|---|---|
| | | | Conc. | | SEC % | IEX % | RPC % | NR % | R % |
| Sample | 40° C. | Repeat | (mg/mL) | pH | Main | Main | Main | Main | Main |
| Formulation 1 | Initial | 1 | 21.94 | 5.81 | 96.39 | 61.15 | 89.18 | 96.41 | 99.77 |
| 20 mg/mL | | 2 | 21.89 | 5.88 | 96.23 | 61.92 | 87.77 | 96.44 | 99.78 |
| | 1 W | 1 | 21.93 | 5.96 | 95.05 | 60.82 | 87.04 | 95.81 | 99.48 |
| | | 2 | 22.05 | 5.97 | 95.13 | 61.21 | 86.88 | 95.88 | 99.47 |
| | 2 W | 1 | 21.95 | 5.96 | 94.37 | 59.32 | 85.88 | 95.19 | 99.15 |
| | | 2 | 22.02 | 5.97 | 94.24 | 59.19 | 86.26 | 95.19 | 98.89 |
| | 4 W | 1 | 21.80 | 5.93 | 92.64 | 55.34 | 86.41 | 94.54 | 99.28 |
| | | 2 | 21.73 | 5.92 | 92.63 | 55.31 | 86.05 | 94.54 | 99.30 |
| Formulation 1 | Initial | 1 | 55.92 | 5.88 | 95.74 | 61.89 | 89.28 | 96.23 | 99.75 |
| 50 mg/mL | | 2 | 56.30 | 5.87 | 96.40 | 61.95 | 87.68 | 96.18 | 99.76 |
| | 1 W | 1 | 55.65 | 5.97 | 95.05 | 61.13 | 86.91 | 95.76 | 99.48 |
| | | 2 | 55.70 | 5.99 | 95.20 | 61.11 | 88.32 | 95.81 | 99.44 |
| | 2 W | 1 | 54.91 | 5.98 | 94.12 | 58.71 | 86.09 | 94.82 | 99.17 |
| | | 2 | 54.96 | 5.98 | 94.11 | 59.32 | 85.87 | 94.85 | 98.95 |
| | 4 W | 1 | 53.49 | 5.93 | 92.45 | 55.58 | 86.21 | 94.51 | 99.07 |
| | | 2 | 52.69 | 5.92 | 92.47 | 55.72 | 86.57 | 94.59 | 99.06 |
| Formulation 1 | Initial | 1 | 89.56 | 5.86 | 96.32 | 61.58 | 89.10 | 96.09 | 99.74 |
| 80 mg/mL | | 2 | 89.86 | 5.82 | 96.09 | 61.38 | 89.55 | 96.04 | 99.75 |
| | 1 W | 1 | 88.74 | 5.97 | 95.09 | 61.19 | 88.53 | 95.73 | 99.46 |
| | | 2 | 88.96 | 5.98 | 94.87 | 61.17 | 86.88 | 95.80 | 99.44 |
| | 2 W | 1 | 87.80 | 5.97 | 93.72 | 59.46 | 85.91 | 94.64 | 98.86 |
| | | 2 | 87.98 | 5.98 | 94.01 | 59.51 | 86.65 | 94.68 | 98.87 |
| | 4 W | 1 | 85.02 | 5.92 | 92.19 | 55.99 | 86.36 | 94.49 | 99.02 |
| | | 2 | 84.11 | 5.93 | 92.26 | 56.03 | 86.34 | 94.52 | 99.02 |
| Formulation 2 | Initial | 1 | 53.49 | 5.73 | 96.69 | 51.78 | 85.03 | 93.88 | 99.70 |
| 50 mg/mL | | 2 | 54.26 | 5.76 | 96.47 | 51.63 | 85.42 | 93.88 | 99.74 |
| | 1 W | 1 | 53.80 | 5.77 | 95.12 | 51.59 | 86.56 | 93.05 | 99.20 |
| | | 2 | 53.63 | 5.76 | 95.24 | 51.17 | 86.57 | 92.98 | 99.04 |
| | 2 W | 1 | 50.37 | 5.73 | 93.81 | 51.48 | 86.32 | 93.14 | 98.66 |
| | | 2 | 52.66 | 5.73 | 93.81 | 51.19 | 86.19 | 93.07 | 98.67 |
| | 4 W | 1 | 55.96 | 5.73 | 91.90 | 47.85 | 83.72 | 92.31 | 96.84 |
| | | 2 | 58.19 | 5.73 | 91.94 | 47.54 | 83.52 | 92.22 | 96.90 |

### −70° C. Freeze-Thaw Cycle Test

The dispensed samples in vials were placed at −70° C. for at least 4 hours to freeze them completely. Afterwards, the samples were thawed completely at room temperature or 25° C. (as one cycle). The changes of each test item were detected by sampling after 1, 3, and 5 cycles, respectively. The results showed that after the freeze-thaw cycles, the samples were colorless and clear liquids without obvious turbidity or precipitation, and there were no significant changes in other test items. Detailed results are shown in the table below.

TABLE 8
Results of formulation confirmation after freeze/thaw cycles

| | | | | | | | | CE-SDS- | CE-SDS- |
|---|---|---|---|---|---|---|---|---|---|
| | Freeze/ | | Conc. | | SEC % | IEX % | RPC % | NR % | R % |
| Sample | thaw | Repeat | (mg/mL) | pH | Main | Main | Main | Main | Main |
| Formulation 1 | Initial | 1 | 21.94 | 5.81 | 96.39 | 62.78 | 89.18 | 96.41 | 99.77 |
| 20 mg/mL | | 2 | 21.89 | 5.88 | 96.23 | 62.78 | 87.77 | 96.44 | 99.78 |
| | 3 | 1 | / | 5.85 | 96.13 | 62.87 | 89.02 | 96.43 | 99.75 |
| | Cycles | 2 | / | 5.83 | 96.27 | 62.81 | 90.48 | 96.26 | 99.73 |
| | 5 | 1 | 21.69 | 5.87 | 96.33 | 62.78 | 89.15 | 96.62 | 99.68 |
| | Cycles | 2 | 21.77 | 5.85 | 96.45 | 63.09 | 89.04 | 96.66 | 99.71 |
| Formulation 1 | Initial | 1 | 55.92 | 5.88 | 95.74 | 62.57 | 89.28 | 96.23 | 99.75 |
| 50 mg/mL | | 2 | 56.30 | 5.87 | 96.40 | 62.69 | 87.68 | 96.18 | 99.76 |
| | 3 | 1 | / | 5.85 | 96.71 | 62.69 | 89.15 | 95.90 | 99.74 |
| | Cycles | 2 | / | 5.85 | 96.44 | 62.75 | 87.78 | 95.94 | 99.73 |
| | 5 | 1 | 54.78 | 5.84 | 96.46 | 63.14 | 87.67 | 96.54 | 99.68 |
| | Cycles | 2 | 54.68 | 5.88 | 96.3 | 62.66 | 87.59 | 96.42 | 99.67 |
| Formulation 1 | Initial | 1 | 89.56 | 5.86 | 96.32 | 62.81 | 89.10 | 96.09 | 99.74 |
| 80 mg/mL | | 2 | 89.86 | 5.82 | 96.09 | 62.64 | 89.55 | 96.04 | 99.75 |
| | 3 | 1 | / | 5.85 | 96.12 | 62.80 | 89.35 | 96.06 | 99.70 |
| | Cycles | 2 | / | 5.82 | 95.94 | 62.80 | 88.38 | 96.14 | 99.70 |
| | 5 | 1 | 88.46 | 5.85 | 96.28 | 62.83 | 89.26 | 96.42 | 99.71 |
| | Cycles | 2 | 88.05 | 5.84 | 96.00 | 62.93 | 87.77 | 96.45 | 99.68 |
| Formulation 2 | Initial | 1 | 53.49 | 5.73 | 96.69 | 52.98 | 85.03 | 93.88 | 99.70 |
| 50 mg/mL | | 2 | 54.26 | 5.76 | 96.47 | 52.86 | 85.42 | 93.88 | 99.74 |
| | 3 | 1 | / | 5.74 | 96.63 | 52.60 | 87.05 | 93.90 | 99.71 |
| | Cycles | 2 | / | 5.74 | 96.51 | 52.73 | 86.51 | 93.96 | 99.77 |
| | 5 | 1 | 55.80 | 5.74 | 96.60 | 52.65 | 87.14 | 93.84 | 99.69 |
| | Cycles | 2 | 55.75 | 5.74 | 96.49 | 52.98 | 86.58 | 93.96 | 99.71 |

### 250 rpm Agitation Test

The dispensed samples were fastened on a shaker in the dark and shaken at 250 rpm The changes of each test item were detected by sampling after 1 day, 3 days, and 5 days, respectively. The results showed that under these agitation conditions, the samples were colorless and clear liquids without obvious turbidity or precipitation. There were no significant changes in other test items. Detailed results are shown in the table below.

TABLE 9
Results of formulation confirmation under agitation conditions

| | | | | | | | | CE- | CE- |
|---|---|---|---|---|---|---|---|---|---|
| | | | | | | | | SDS- | SDS- |
| | | | Conc. | | SEC % | IEX % | RPC % | NR % | R % |
| Sample | Shake | Repeat | (mg/mL) | PH | Main | Main | Main | Main | Main |
| Formulation 1 | Initial | 1 | 21.94 | 5.81 | 96.39 | 62.78 | 89.18 | 96.41 | 99.77 |
| 20 mg/mL | | 2 | 21.89 | 5.88 | 96.23 | 62.78 | 87.77 | 96.44 | 99.78 |
| | 1 day | 1 | / | 5.85 | 96.32 | 62.36 | 89.18 | 96.67 | 99.71 |
| | | 2 | / | 5.81 | 96.61 | 62.85 | 87.6 | 96.48 | 99.67 |
| | 3 days | 1 | / | 5.95 | 96.64 | 62.69 | 87.25 | 96.54 | 99.70 |
| | | 2 | / | 5.96 | 96.39 | 62.56 | 87.72 | 96.48 | 99.70 |
| | 5 days | 1 | 22.07 | 5.95 | 96.64 | 62.20 | 87.75 | 96.56 | 99.59 |
| | | 2 | 21.98 | 5.94 | 96.60 | 62.22 | 88.01 | 96.46 | 99.62 |
| Formulation 1 | Initial | 1 | 55.92 | 5.88 | 95.74 | 62.57 | 89.28 | 96.23 | 99.75 |
| 50 mg/mL | | 2 | 56.30 | 5.87 | 96.40 | 62.69 | 87.68 | 96.18 | 99.76 |
| | 1 day | 1 | / | 5.84 | 96.26 | 62.65 | 89.29 | 96.26 | 99.69 |
| | | 2 | / | 5.86 | 96.17 | 62.74 | 87.64 | 96.06 | 99.68 |
| | 3 days | 1 | / | 5.95 | 96.35 | 62.83 | 89.07 | 96.24 | 99.78 |
| | | 2 | / | 5.98 | 96.22 | 62.62 | 89.13 | 96.28 | 99.76 |
| | 5 days | 1 | 55.76 | 5.97 | 96.33 | 62.50 | 87.48 | 96.40 | 99.56 |
| | | 2 | 55.58 | 5.96 | 96.33 | 62.50 | 87.72 | 96.39 | 99.58 |
| Formulation 1 | Initial | 1 | 89.56 | 5.86 | 96.32 | 62.81 | 89.10 | 96.09 | 99.74 |
| 80 mg/mL | | 2 | 89.86 | 5.82 | 96.09 | 62.64 | 89.55 | 96.04 | 99.75 |
| | 1 day | 1 | / | 5.85 | 96.13 | 62.80 | 89.31 | 96.42 | 99.70 |
| | | 2 | / | 5.83 | 96.30 | 63.00 | 89.17 | 96.42 | 99.45 |
| | 3 days | 1 | / | 5.98 | 95.92 | 62.61 | 88.44 | 96.24 | 99.66 |
| | | 2 | / | 5.99 | 96.16 | 62.67 | 89.03 | 96.25 | 99.79 |
| | 5 days | 1 | 88.80 | 5.97 | 96.46 | 62.32 | 87.82 | 96.03 | 99.65 |
| | | 2 | 89.19 | 5.96 | 96.18 | 62.76 | 89.30 | 96.07 | 99.59 |
| Formulation 2 | Initial | 1 | 53.49 | 5.73 | 96.69 | 52.98 | 85.03 | 93.88 | 99.70 |
| 50 mg/mL | | 2 | 54.26 | 5.76 | 96.47 | 52.86 | 85.42 | 93.88 | 99.74 |
| | 1 day | 1 | / | 5.75 | 96.69 | 52.86 | 86.65 | 94.02 | 99.76 |
| | | 2 | / | 5.75 | 96.39 | 52.64 | 87.16 | 93.84 | 99.73 |
| | 3 days | 1 | / | 5.75 | 96.75 | 52.64 | 86.60 | 93.67 | 99.79 |
| | | 2 | / | 5.75 | 96.59 | 53.04 | 86.93 | 93.89 | 99.69 |
| | 5 days | 1 | 55.15 | 5.78 | 96.10 | 52.75 | 85.64 | 93.88 | 99.79 |
| | | 2 | 54.84 | 5.76 | 96.42 | 52.48 | 85.58 | 93.92 | 97.89 |

### 25° C. Incubation Test

The protein samples were placed in a constant temperature incubator at 25° C. in the dark for 6 months. The changes of each test item (e.g., appearance, protein concentration, pH, purity (detected by SEC-HPLC, RPC-UPLC, and CE-SDS methods), and charge isomers (detected by IEX-HPLC)) were detected by sampling at 0 month, 1 month, 2 months, 3 months, and 6 months, respectively. The results showed that under these conditions, the samples were colorless and clear liquids without obvious turbidity or precipitation. Detailed results are shown in the table below. The results showed that the change of protein concentration within 6 months at 25° C. was less than 10%, the decrease of SEC-HPLC purity was less than 3.5% within 6 months at 25° C., and the main peak ratio of charge isomers decreased by less than 15% within 6 months at 25° C. Within 6 months at 25° C., the purity of non-reduced and reduced CE-SDS decreased by less than 3%.

TABLE 10
Results of formulation confirmation at 25° C.

| | | | | | | | | CE- | CE- |
|---|---|---|---|---|---|---|---|---|---|
| | | | | | | | | SDS- | SDS- |
| | | | Conc. | | SEC % | IEX % | RP % | NR % | R % |
| Sample | 25° C. | Repeat | (mg/ml) | pH | Main | Main | Main | Main | Main |
| Formulation 1 | Initial | 1 | 21.94 | 5.81 | 96.39 | 61.15 | 89.18 | 96.41 | 99.77 |
| 20 mg/ml | | 2 | 21.89 | 5.88 | 96.23 | 61.92 | 87.77 | 96.44 | 99.78 |
| | 1 M | 1 | 21.57 | 5.89 | 95.42 | 61.22 | 86.41 | 96.22 | 99.46 |
| | | 2 | 21.73 | 5.90 | 95.29 | 61.05 | 86.05 | 96.30 | 99.47 |
| | 2 M | 1 | 21.17 | 5.88 | 95.12 | 59.48 | 85.26 | 95.96 | 99.06 |
| | | 2 | 21.50 | 5.86 | 95.38 | 59.56 | 86.83 | 96.01 | 99.05 |
| | 3 M | 1 | 21.47 | 5.94 | 94.89 | 59.63 | 83.44 | 95.69 | 98.77 |
| | | 2 | 21.41 | 5.89 | 94.95 | 59.67 | 83.54 | 95.71 | 98.77 |
| | 6 M | 1 | 22.08 | 5.88 | 94.18 | 48.86 | 80.63 | 94.57 | 97.95 |
| | | 2 | 22.10 | 5.90 | 94.12 | 48.10 | 80.38 | 94.33 | 97.95 |
| Formulation 1 | Initial | 1 | 55.92 | 5.88 | 95.74 | 61.89 | 89.28 | 96.23 | 99.75 |
| 50 mg/ml | | 2 | 56.30 | 5.87 | 96.40 | 61.95 | 87.68 | 96.18 | 99.76 |
| | 1 M | 1 | 53.46 | 5.90 | 95.21 | 61.12 | 86.21 | 96.04 | 99.44 |
| | | 2 | 53.79 | 5.92 | 95.26 | 61.40 | 86.57 | 96.16 | 99.41 |
| | 2 M | 1 | 54.08 | 5.86 | 95.20 | 59.37 | 87.00 | 95.84 | 99.08 |
| | | 2 | 52.13 | 5.87 | 95.07 | 59.93 | 86.07 | 96.04 | 99.01 |
| | 3 M | 1 | 53.81 | 5.89 | 94.85 | 59.83 | 85.99 | 95.44 | 98.75 |
| | | 2 | 53.46 | 5.91 | 94.73 | 59.87 | 84.97 | 95.45 | 98.78 |
| | 6 M | 1 | 53.60 | 5.93 | 94.13 | 48.71 | 80.98 | 94.42 | 97.71 |
| | | 2 | 54.05 | 5.90 | 94.08 | 48.76 | 79.84 | 94.15 | 97.73 |
| Formulation 1 | Initial | 1 | 89.56 | 5.86 | 96.32 | 61.58 | 89.10 | 96.09 | 99.74 |
| 80 mg/ml | | 2 | 89.86 | 5.82 | 96.09 | 61.38 | 89.55 | 96.04 | 99.75 |
| | 1 M | 1 | 86.70 | 5.92 | 95.23 | 61.17 | 86.36 | 95.99 | 99.41 |
| | | 2 | 84.28 | 5.92 | 95.22 | 61.60 | 86.34 | 96.02 | 99.42 |
| | 2 M | 1 | 82.55 | 5.88 | 95.06 | 59.60 | 85.95 | 96.03 | 98.93 |
| | | 2 | 83.09 | 5.88 | 95.04 | 59.92 | 86.78 | 96.08 | 98.87 |
| | 3 M | 1 | 87.07 | 5.92 | 94.80 | 59.82 | 85.48 | 95.38 | 98.73 |
| | | 3 | 83.95 | 5.89 | 94.68 | 59.86 | 83.70 | 95.33 | 98.74 |
| | 6 M | 1 | 88.88 | 5.89 | 93.93 | 49.05 | 80.08 | 94.45 | 97.56 |
| | | 2 | 89.66 | 5.92 | 93.99 | 49.46 | 80.55 | 94.31 | 97.68 |
| Formulation 2 | Initial | 1 | 53.49 | 5.73 | 96.69 | 51.78 | 85.03 | 93.88 | 99.70 |
| 50 mg/ml | | 2 | 54.26 | 5.76 | 96.47 | 51.63 | 85.42 | 93.88 | 99.74 |
| | 1 M | 1 | 56.22 | 5.75 | 96.03 | 51.10 | 85.51 | 93.40 | 99.45 |
| | | 2 | 56.10 | 5.74 | 96.21 | 50.75 | 85.45 | 93.39 | 99.46 |
| | 2 M | 1 | 52.04 | 5.67 | 95.60 | 50.21 | 84.29 | 93.23 | 98.81 |
| | | 2 | 51.75 | 5.65 | 95.85 | 49.73 | 83.62 | 93.26 | 98.71 |
| | 3 M | 1 | 51.59 | 5.74 | 95.07 | 51.93 | 81.95 | 92.92 | 98.33 |
| | | 2 | 51.21 | 5.71 | 95.06 | 52.05 | 82.35 | 92.92 | 98.66 |
| | 6 M | 1 | 57.56 | 5.68 | 93.35 | 42.17 | 77.50 | 91.91 | 97.27 |
| | | 2 | 57.54 | 5.67 | 93.55 | 41.80 | 77.44 | 91.95 | 97.36 |

In conclusion, all tested BH3120 formulations showed good thermal stability. As expected, the experimental results show that the formulations can better inhibit the formation of acid peaks.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of claims.

The sequences disclosed in the description are shown in Table 11.

TABLE 11
Sequences disclosed in the description

| SEQ ID NO | Description | Sequence |
|---|---|---|
| 1 | Anti-human PD-L1 | GACATCCAGATGACCCAGTCCCCTTCCTCCCTGTCCGCCTCTGTGGGC |
| | antibody light chain | GACAGGGTGACCATCACCTGCAAGGCCTCCCAGGATGTGCACACCG |
| | variable region | CCGTGGCTTGGATTCAGCAGAAGCCCGGCAAGTCCCCCAAGCTGCTG |
| | (nucleotide sequence) | ATCTACTCCGCCTCCAACCGGTACACCGGCGTGCCTAGCAGGTTCAG |
| | | CGGAAGCGGCAGCGGCACCGACTTCACCTTCACCATCTCCTCCCTGC |
| | | AGCCTGAGGACATCGCCACCTATTACTGCCAGCAGCACTACATCACC |
| | | CCCCTGACCTTCGGCCAGGGCACCAAGCTGGAGATCAAG |
| 2 | Anti-human PD-L1 | DIQMTQSPSSLSASVGDRVTITCKASQDVHTAVAWIQQKPGKSPKLLIYS |
| | antibody light chain | ASNRYTGVPSRFSGSGSGTDFTFTISSLQPEDIATYYCQQHYITPLTFGQG |
| | variable region | TKLEIK |
| | (amino acid | |
| | sequence) | |
| 3 | Light chain constant | CGTACGGTGGCCGCCCCCAGCGTCTTCATTTTTCCACCCTCTGACGAA |
| | region (nucleotide | CAGCTGAAGTCAGGGACAGCTTCCGTGGTCTGTCTGCTGAACAATTTT |
| | sequence) | TACCCCAGGGAGGCCAAAGTGCAGTGGAAGGTCGATAACGCTCTGCA |
| | | GAGCGGAAATTCTCAGGAGAGTGTGACAGAACAGGACTCAAAAGAT |
| | | TCCACTTATAGCCTGTCTAGTACCCTGACACTGTCCAAGGCAGACTAC |
| | | GAAAAGCATAAAGTGTATGCCTGTGAGGTCACACATCAGGGTCTGTC |
| | | AAGCCCCGTCACTAAGTCCTTCAACCGGGGAGAATGC |
| 4 | Light chain constant | RTVAAPSVFIFPPSDEQLKSGTASVVCLLNNFYPREAKVQWKVDNALQS |
| | region (amino acid | GNSQESVTEQDSKDSTYSLSSTLTLSKADYEKHKVYACEVTHQGLSSPV |
| | sequence) | TKSFNRGEC |
| 5 | Anti-human PD-L1 | GAAGTGCAGCTGGTGCAGTCCGGCGCCGAGGTGAAGAAGCCTGGCT |
| | antibody heavy chain | CCTCCGTGAAGGTGTCCTGCAAGGCCTCCGGCTTCAACATCGAGGAC |
| | variable region | ACCTACATCCACTGGGTGAGGCAGGCTCCCGGACAGGGACTGGAGT |
| | (nucleotide sequence) | GGATGGGCAGGATCGATCCCGCCAACGCCAACACCAAGTACGACCC |
| | | CAAGTTCCAGGGCAGGGTGACCATCACCGCCGATACCAGCACCAAC |
| | | ACCGCCTACATGGAGCTGTCCTCCCTGAGGTCCGAGGACACAGCCGT |
| | | GTACTACTGTGGCAGGGGACTGGGCGCCTGGTTTGCCTACTGGGGAC |
| | | AGGGCACCCTGGTGACCGTGTCCTCC |
| 6 | Anti-human PD-L1 | EVQLVQSGAEVKKPGSSVKVSCKASGFNIEDTYIHWVRQAPGQGLEW |
| | antibody heavy chain | MGRIDPANANTKYDPKFQGRVTITADTSTNTAYMELSSLRSEDTAVYYC |
| | variable region | GRGLGAWFAYWGQGTLVTVSS |
| | (amino acid | |
| | sequence) | |
| 7 | Anti-human PD-L1 | GCTAGCACAAAAGGACCTTCCGTGTTTCCACTGGCACCCTCTAGTAAG |
| | antibody heavy chain | AGTACTTCAGGAGGAACCGCAGCACTGGGATGTCTGGTGAAGGACTA |
| | constant region | CTTCCCAGAGCCCGTCACCGTGTCTTGGAACAGTGGAGCACTGACCT |
| | (nucleotide sequence) | CCGGGGTCCATACATTTCCTGCCGTGCTGCAGTCATCCGGTCTGTATAG |
| | | CCTGAGCTCTGTGGTCACAGTCCCAAGTTCATCCCTGGGCACCCAGA |
| | | CATACATCTGCAACGTGAATCACAAACCTTCCAATACTAAGGTCGACA |
| | | AGAAAGTGGAACCCAAGTCCTGCGATAAGACCCACACATGCCCTCCC |
| | | TGTCCTGCTCCCGAACTGCTGGGAGGACCCTCCGTCTTCCTGTTCCCC |
| | | CCCAAGCCCAAAGACACACTGATGATCAGCAGGACCCCTGAAGTGAC |
| | | CTGCGTGGTCGTGGACGTGAGCCACGAGGACCCCGAGGTCAAGTTTA |
| | | ACTGGTACGTGGACGGCGTGGAGGTCCACAACGCCAAGACCAAGCC |
| | | CAGGGAGGAGCAGTACAACAGCACCTACAGGGTCGTGTCCGTGCTGA |
| | | CCGTGCTCCACCAAGATTGGCTCAACGGCAAGGAGTATAAGTGCAAA |
| | | GTCAGCAACAAGGCCCTCCCCGCCCCCATCGAGAAAACCATCAGCAA |
| | | GGCCAAGGGCCAACCGCGGGAACCTCAAGTGTATACCCTCCCTCCCA |
| | | GCCGGGATGAGCTGACCAAGAACCAAGTCTCCCTCTTGTGCCTGGTC |
| | | AAGGGATTCTACCCTTCCGACATTGCCGTCGAATGGGAGAGCAATGGC |
| | | CAGCCCGAGAACAACTACAAGACAACCCCCCCCGTCCTGCGCAGCGA |
| | | CGGATCCTTCTTCCTGTACTCCAAGCTCACCGTGGACAAGAGCCGGTG |
| | | GCAACAGGGCAACGTGTTCTCCTGTAGCGTGATGCACGAAGCCCTCC |
| | | ACAACCACTATACCCAGAAGAGCCTGAGCCTCAGCCCCGGCAAA |
| 8 | Anti-human PD-L1 | ASTKGPSVFPLAPSSKSTSGGTAALGCLVKDYFPEPVTVSWNSGALTSG |
| | antibody heavy chain | VHTFPAVLQSSGLYSLSSVVTVPSSSLGTQTYICNVNHKPSNTKVDKKVE |
| | constant region | PKSCDKTHTCPPCPAPELLGGPSVFLFPPKPKDTLMISRTPEVTCVVVDV |
| | (amino acid | SHEDPEVKFNWYVDGVEVHNAKTKPREEQYNSTYRVVSVLTVLHQDW |
| | sequence) | LNGKEYKCKVSNKALPAPIEKTISKAKGQPREPQVYTLPPSRDELTKNQ |
| | | VSLLCLVKGFYPSDIAVEWESNGQPENNYKTTPPVLRSDGSFFLYSKLTV |
| | | DKSRWQQGNVFSCSVMHEALHNHYTQKSLSLSPGK |
| 9 | Anti-human 4-1BB | GACATCCAGATGACCCAGAGCCCTAGCAGCCTGAGCGCCAGCGTGG |
| | antibody light chain | GCGACAGAGTGACCATCACCTGCAGAGCCAGCCAGGACATCAGCAA |
| | variable region | TTACCTGAATTGGTACCAGCAGAAGCCTGGCAAGGTGCCTAAGCTGC |
| | (nucleotide sequence) | TGATCTACCACACCAGCAGACTGCACAGCGGCGTGCCTAGCAGATTC |
| | | AGCGGCAGCGGCAGCGGCACCGACTTCACCCTGACCATCAGCAGCC |
| | | TGCAGCCTGAGGACGTGGCCACCTACTACTGCCAGCAGGGCCACAC |
| | | CCTGCCTAGAACCTTCGGCGGCGGCACCAAGGTGGAGATCAAG |
| 10 | Anti-human 4-1BB | DIQMTQSPSSLSASVGDRVTITCRASQDISNYLNWYQQKPGKVPKLLIY |
| | antibody light chain | HTSRLHSGVPSRFSGSGSGTDFTLTISSLQPEDVATYYCQQGHTLPRTFG |
| | variable region | GGTKVEIK |
| | (amino acid | |
| | sequence) | |
| 11 | Anti-human 4-1BB | CAGGTGCAGCTGGTGCAGAGCGGCGCCGAGGTGAAGAAGCCTGGCA |
| | antibody heavy chain | GCAGCGTGAAGGTGAGCTGCAAGGCCAGCGGCTACACCTTCACCAG |
| | variable region | CTACTGGATCAATTGGGTGAGACAGGCTCCGGGCCAAGGGCTGGAG |
| | (nucleotide sequence) | TGGATGGGCAATATCTACCCTAGCGACAGCTACACCAATTACAATCAG |
| | | AAGTTCAAGGACAGAGTGACCATCACCGCCGACAAGAGCACCAGCA |
| | | CCGCCTACATGGAGCTGAGCAGCCTGAGAAGCGAGGACACCGCCGT |
| | | GTACTACTGCGCCAGACTGTACTACGGCAGCAGCCCTTTCGACTACT |
| | | GGGGACAAGGCACCACCGTGACCGTGAGCAGC |
| 12 | Anti-human 4-1BB | QVQLVQSGAEVKKPGSSVKVSCKASGYTFTSYWINWVRQAPGQGLEW |
| | antibody heavy chain | MGNIYPSDSYTNYNQKFKDRVTITADKSTSTAYMELSSLRSEDTAVYYC |
| | variable region | ARLYYGSSPFDYWGQGTTVTVSS |
| | (amino acid | |
| | sequence) | |
| 13 | Anti-human 4-1BB | GCTAGCACAAAAGGACCTTCCGTGTTTCCACTGGCACCCTCTAGTAA |
| | antibody heavy chain | GAGTACTTCAGGAGGAACCGCAGCACTGGGATGTCTGGTGAAGGAC |
| | constant region | TACTTCCCAGAGCCCGTCACCGTGTCTTGGAACAGTGGAGCACTGAC |
| | (nucleotide sequence) | CTCCGGGGTCCATACATTTCCTGCCGTGCTGCAGTCATCCGGTCTGTA |
| | | TAGCCTGAGCTCTGTGGTCACAGTCCCAAGTTCATCCCTGGGCACCC |
| | | AGACATACATCTGCAACGTGAATCACAAACCTTCCAATACTAAGGTC |
| | | GACAAGAAAGTGGAACCCAAGTCCTGCGATAAGACCCACACATGCC |
| | | CTCCCTGTCCTGCTCCCGAACTGCTGGGAGGACCCTCCGTCTTCCTG |
| | | TTCCCCCCCAAGCCCAAAGACACACTGATGATCAGCAGGACCCCTGA |
| | | AGTGACCTGCGTGGTCGTGGACGTGAGCCACGAGGACCCCGAGGTC |
| | | AAGTTTAACTGGTACGTGGACGGCGTGGAGGTCCACAACGCCAAGA |
| | | CCAAGOCCAGGGAGGAGCAGTACGCCAGCACCTACAGGGTGGTCAG |
| | | CGTGCTGACCGTGCTGCACCAGGATTGGCTCAACGGCAAGGAGTAC |
| | | AAGTGCAAAGTCTCCAACAAGGCCCTGCCCGCCCCCATCGAGAAGA |
| | | CCATCTCCAAGGCTAAGGGACAGCCCAGGGAGCCCCAAGTGTACAC |
| | | CGAGCCTCCCAGCCGGGATGAGCTGACCAAGAACCAAGTCTCCCTC |
| | | ACCTGCCTGGTCAAGGGATTCTACCCTTCCGACATTGCCGTCGAATG |
| | | GGAGAGCAATGGCCAGCCCGAGAACAACTACAAGACAACCCCCCCC |
| | | GTCCTGGATAGCGACGGATCCTTCTTCCTGCTCTCCGTGCTCACCGTC |
| | | GACAAGAGCAGATGGCAGCAGGGCAACGTGTTCAGCTGTAGCGTGA |
| | | TGCACGAGGCCCTGCACAACCACTACACCCAGAAGAGCCTGTCCCT |
| | | CAGCCCCGGCAAG |
| 14 | Anti-human 4-1BB | ASTKGPSVFPLAPSSKSTSGGTAALGCLVKDYFPEPVTVSWNSGALTSG |
| | antibody heavy chain | VHTFPAVLQSSGLYSLSSVVTVPSSSLGTQTYICNVNHKPSNTKVDKKVE |
| | constant region | PKSCDKTHTCPPCPAPELLGGPSVFLFPPKPKDTLMISRTPEVTCVVVDV |
| | (amino acid | SHEDPEVKFNWYVDGVEVHNAKTKPREEQYASTYRVVSVLTVLHQDW |
| | sequence) | LNGKEYKCKVSNKALPAPIEKTISKAKGQPREPQVYTEPPSRDELTKNQ |
| | | VSLTCLVKGFYPSDIAVEWESNGQPENNYKTTPPVLDSDGSFFLLSVLTV |
| | | DKSRWQQGNVFSCSVMHEALHNHYTQKSLSLSPGK |
| 15 | Anti-human PD-L1 | GFNIEDTYIH |
| | antibody heavy chain | |
| | variable region | |
| | HCDR1 | |
| 16 | Anti-human PD-L1 | RIDPANANTKYDPKFQG |
| | antibody heavy chain | |
| | variable region | |
| | HCDR2 | |
| 17 | Anti-human PD-L1 | GLGAWFAY |
| | antibody heavy chain | |
| | variable region | |
| | HCDR3 | |
| 18 | Anti-human PD-L1 | KASQDVHTAVA |
| | antibody light chain | |
| | variable region | |
| | LCDR1 | |
| 19 | Anti-human PD-L1 | SASNRYT |
| | antibody light chain | |
| | variable region | |
| | LCDR2 | |
| 20 | Anti-human PD-L1 | QQHYITPLT |
| | antibody light chain | |
| | variable region | |
| | LCDR3 | |
| 21 | Anti-human 4-1BB | GYTFTSYWIN |
| | antibody heavy chain | |
| | variable region | |
| | HCDR1 | |
| 22 | Anti-human 4-1BB | NIYPSDSYTNYNQKFKD |
| | antibody heavy chain | |
| | variable region | |
| | HCDR2 | |
| 23 | Anti-human 4-1BB | LYYGSSPFDY |
| | antibody heavy chain | |
| | variable region | |
| | HCDR3 | |
| 24 | Anti-human 4-1BB | RASQDISNYLN |
| | antibody light chain | |
| | variable region | |
| | LCDR1 | |
| 25 | Anti-human 4-1BB | HTSRLHS |
| | antibody light chain | |
| | variable region | |
| | LCDR2 | |
| 26 | Anti-human 4-1BB | QQGHTLPRT |
| | antibody light chain | |
| | variable region | |
| | LCDR3 | |
| 27 | Anti-human PD-L1 | EVQLVQSGAEVKKPGSSVKVSCKASGFNIEDTYIHWVRQAPGQGLEW |
| | antibody heavy chain | MGRIDPANANTKYDPKFQGRVTITADTSTNTAYMELSSLRSEDTAVYYC |
| | (amino acid | GRGLGAWFAYWGQGTLVTVSSASTKGPSVFPLAPSSKSTSGGTAALGCL |
| | sequence) | VKDYFPEPVTVSWNSGALTSGVHTFPAVLQSSGLYSLSSVVTVPSSSLGT |
| | | QTYICNVNHKPSNTKVDKKVEPKSCDKTHTCPPCPAPELLGGPSVFLFPP |
| | | KPKDTLMISRTPEVTCVVVDVSHEDPEVKFNWYVDGVEVHNAKTKPR |
| | | EEQYNSTYRVVSVLTVLHQDWLNGKEYKCKVSNKALPAPIEKTISKAK |
| | | GQPREPQVYTLPPSRDELTKNQVSLLCLVKGFYPSDIAVEWESNGQPEN |
| | | NYKTTPPVLRSDGSFFLYSKLTVDKSRWQQGNVFSCSVMHEALHNHYT |
| | | QKSLSLSPGK |
| 28 | Anti-human PD-L1 | DIQMTQSPSSLSASVGDRVTITCKASQDVHTAVAWIQQKPGKSPKLLIYS |
| | antibody light chain | ASNRYTGVPSRFSGSGSGTDFTFTISSLQPEDIATYYCQQHYITPLTFGQG |
| | (amino acid | TKLEIKRTVAAPSVFIFPPSDEQLKSGTASVVCLLNNFYPREAKVQWKV |
| | sequence) | DNALQSGNSQESVTEQDSKDSTYSLSSTLTLSKADYEKHKVYACEVTH |
| | | QGLSSPVIKSFNRGEC |
| 29 | Anti-human 4-1BB | QVQLVQSGAEVKKPGSSVKVSCKASGYTFTSYWINWVRQAPGQGLEW |
| | antibody heavy chain | MGNIYPSDSYTNYNQKFKDRVTITADKSTSTAYMELSSLRSEDTAVYYC |
| | (amino acid | ARLYYGSSPFDYWGQGTTVTVSSASTKGPSVFPLAPSSKSTSGGTAALG |
| | sequence) | CLVKDYFPEPVTVSWNSGALTSGVHTFPAVLQSSGLYSLSSVVTVPSSSL |
| | | GTQTYICNVNHKPSNTKVDKKVEPKSCDKTHTCPPCPAPELLGGPSVFL |
| | | FPPKPKDTLMISRTPEVTCVVVDVSHEDPEVKFNWYVDGVEVHNAKTK |
| | | PREEQYASTYRVVSVLTVLHQDWLNGKEYKCKVSNKALPAPIEKTISKA |
| | | KGQPREPQVYTEPPSRDELTKNQVSLTCLVKGFYPSDIAVEWESNGQPE |
| | | NNYKTTPPVLDSDGSFFLLSVLTVDKSRWQQGNVFSCSVMHEALHNHY |
| | | TQKSLSLSPGK |
| 30 | Anti-human 4-1BB | DIQMTQSPSSLSASVGDRVTITCRASQDISNYLNWYQQKPGKVPKLLIY |
| | antibody light chain | HTSRLHSGVPSRFSGSGSGTDFTLTISSLQPEDVATYYCQQGHTLPRTFG |
| | (amino acid | GGTKVEIKRTVAAPSVFIFPPSDEQLKSGTASVVCLLNNFYPREAKVQW |
| | sequence) | KVDNALQSGNSQESVTEQDSKDSTYSLSSTLTLSKADYEKHKVYACEV |
| | | THQGLSSPVTKSFNRGEC |

1. A stable aqueous pharmaceutical formulation comprising
an anti-PD-L1/anti-4-1BB bispecific antibody or antigen-binding fragment thereof; and
a buffer, wherein the formulation has a pH of about 5.0 to about 6.5.

2. The formulation of claim 1, wherein the formulation has a pH of about 5.5 to about 6.0:
preferably, the formulation has a pH of about 5.6 to about 5.8 or about 5.8 to about 6.0.

3. (canceled)

4. The formulation of claim 1, wherein the anti-PD-L1/anti-4-1BB bispecific antibody or antigen-binding fragment thereof can bind to a human PD-L1 and/or a human 4-1BB.

5. The formulation of claim 1, wherein the anti-PD-L1/anti-4-1BB bispecific antibody or antigen-binding fragment thereof has a concentration of about 10 mg/ml to about 100 mg/ml;
preferably, the anti-PD-L1/anti-4-1BB bispecific antibody or antigen-binding fragment thereof has a concentration of about 20 mg/ml to about 80 mg/ml;
preferably, the anti-PD-L1/anti-4-1BB bispecific antibody or antigen-binding fragment thereof has a concentration of about 50 mg/ml.

6.-7. (canceled)

8. The formulation of claim 1, wherein the buffer comprises a buffering agent selected from the group consisting of acetic acid, sodium acetate, tartrate, hydrogen chloride, sodium dihydrogen phosphate, and combinations thereof;
preferably, the buffer comprises acetic acid and/or sodium acetate;
preferably, the formulation comprises an acetate acid/sodium acetate buffer with a concentration of about 10 mM to about 100 mM;
preferably, the formulation comprises an acetate acid/sodium acetate buffer with a concentration of about 10 or about 15 mM.

9.-11. (canceled)

12. The formulation of claim 1, further comprising a tonicity agent selected from the group consisting of trehalose, sucrose, proline, glycine, arginine, alanine, glutamate, methionine, sodium chloride, potassium chloride, magnesium chloride, sodium sulfate and combinations thereof;
preferably, the tonicity agent is trehalose, arginine, or a combination thereof.

13. (canceled)

14. The formulation of claim 1, wherein the formulation comprises from about 10 mM to about 200 mM of arginine;
preferably, the formulation comprises about 100 mM or about 140 mM of arginine;
preferably, the arginine is the hydrochloride salt form of arginine (arginine-HCl).

15.-16. (canceled)

17. The formulation of claim 1, wherein the formulation comprises from about 10 mM to about 300 mM of trehalose;
preferably, the formulation comprises about 130 mM of trehalose.

18. (canceled)

19. The formulation of claim 1, further comprising a surfactant selected from the group consisting of polysorbate 20, polysorbate 40, polysorbate 60, polysorbate 80, SDS, poloxamer 188 (Pluronic® F68), and combinations thereof;
preferably, the surfactant is polysorbate 80.

20. (canceled)

21. The formulation of claim 1, wherein the formulation comprises from about 0.01 mg/ml to about 10 mg/ml polysorbate 80;
preferably, the formulation comprises about 0.2 mg/ml polysorbate 80.

22. (canceled)

23. The formulation of claim 1, wherein the formulation comprises:
an anti-PD-L1/anti-4-1BB bispecific antibody or antigen-binding fragment thereof with a concentration of about 10 mg/ml to about 100 mg/ml;
a tonicity agent with a concentration of about 100 mM to about 300 mM;
a surfactant with a concentration of about 0.01 mg/ml-about 10 mg/ml; and
a buffer system, wherein the formulation has a pH of about 5.0 to about 6.5;
preferably, the formulation has a pH of about 5.5 to about 6.0.

24. (canceled)

25. The formulation of claim 23, wherein the buffer system comprises one or more buffering agents selected from the group consisting of acetic acid, sodium acetate, tartrate, hydrogen chloride, and sodium dihydrogen phosphate;
preferably, the buffer system comprises acetic acid/sodium acetate buffer with a concentration of about 10 to about 100 mM.

26. (canceled)

27. The formulation of claim 23, wherein the tonicity agent is trehalose, sucrose, proline, glycine, arginine, alanine, glutamate, methionine, sodium chloride, potassium chloride, magnesium chloride, sodium sulfate, or combinations thereof;
preferably, the tonicity agent is trehalose, arginine (e.g. arginine-HCl), or a combination thereof.

28. (canceled)

29. The formulation of claim 23, wherein the surfactant is polysorbate 20, polysorbate 40, polysorbate 60, polysorbate 80, SDS, poloxamer 188 (Pluronic® F68), or combinations thereof;
preferably, the surfactant is polysorbate 80.

30. (canceled)

31. The formulation of claim 1, wherein the formulation comprises or consists of:
an anti-PD-L1/anti-4-1BB bispecific antibody or antigen-binding fragment thereof with a concentration of about 10 mg/ml to about 100 mg/ml;
arginine (e.g., arginine-HCl) with a concentration of about 10 mM to about 200 mM;
optionally trehalose with a concentration of about 10 mM to about 300 mM;
polysorbate 80 with a concentration of about 0.01 mg/ml to about 10 mg/ml; and
acetic acid/sodium acetate buffer with a concentration of about 10 to about 100 mM,
wherein the formulation has a pH of about 5.5 to about 6.0;
preferably, the concentration of the anti-PD-L1/anti-4-1BB bispecific antibody or antigen-binding fragment thereof is about 20 mg/ml, about 50 mg/ml, or about 80 mg/ml;
preferably, the concentration of arginine is about 100 mM or about 140 mM;
preferably, the concentration of trehalose is about 130 mM;
preferably, the concentration of polysorbate 80 is about 0.2 mg/ml;
preferably, the concentration of acetic acid/sodium acetate buffer is about 10 mM or about 15 mM.

32.-36. (canceled)

37. The formulation of claim 1, wherein the anti-PD-L1/anti-4-1BB bispecific antibody or antigen-binding fragment thereof comprises:
a first heavy chain variable region (VH1) comprising complementarity determining regions (CDRs) 1, 2, and 3, wherein the VH1 CDR1 region comprises an amino acid sequence that is at least 80% identical to SEQ ID NO:15, the VH1 CDR2 region comprises an amino acid sequence that is at least 80% identical to SEQ ID NO:16, and the VH1 CDR3 region comprises an amino acid sequence that is at least 80% identical to SEQ ID NO:17;
a first light chain variable region (VL1) comprising CDRs 1, 2, and 3, wherein the VL1 CDR1 region comprises an amino acid sequence that is at least 80% identical to SEQ ID NO:18, the VL1 CDR2 region comprises an amino acid sequence that is at least 80% identical to SEQ ID NO:19, and the VL1 CDR3 region comprises an amino acid sequence that is at least 80% identical to SEQ ID NO:20,
wherein the VH1 and VL1 can interact with each other, forming an antigen-binding site that binds to PD-L1.

38. The formulation of claim 1, wherein the anti-PD-L1/anti-4-1BB bispecific antibody or antigen-binding fragment thereof comprises:
a second heavy chain variable region (VH2) comprising complementarity determining regions (CDRs) 1, 2, and 3, wherein the VH2 CDR1 region comprises an amino acid sequence that is at least 80% identical to SEQ ID NO:21, the VH2 CDR2 region comprises an amino acid sequence that is at least 80% identical to SEQ ID NO:22, and the VH2 CDR3 region comprises an amino acid sequence that is at least 80% identical to SEQ ID NO:23; and
a second light chain variable region (VL2) comprising CDRs 1, 2, and 3, wherein the VL2 CDR1 region comprises an amino acid sequence that is at least 80% identical to SEQ ID NO:24, the VL2 CDR2 region comprises an amino acid sequence that is at least 80% identical to SEQ ID NO:25, and the VL2 CDR3 region comprises an amino acid sequence that is at least 80% identical to SEQ ID NO:26,
wherein the VH2 and VL2 can interact with each other, forming an antigen-binding site that binds to 4-1BB.

39. The formulation of claim 1, wherein the anti-PD-L1/anti-4-1BB bispecific antibody or antigen-binding fragment thereof comprises:
a first heavy chain variable region (VH1) comprising an amino acid sequence that is at least 90% identical to SEQ ID NO:6, and a first light chain variable region (VL1) comprising an amino acid sequence that is at least 90% identical to SEQ ID NO:2, wherein the VH1 and VL1 can interact with each other, forming an antigen-binding site that binds to PD-L1, and
a second heavy chain variable region (VH2) comprising an amino acid sequence that is at least 90% identical to SEQ ID NO:12, and a second light chain variable region (VL2) comprising an amino acid sequence that is at least 90% identical to SEQ ID NO:10, wherein the VH2 and VL2 can interact with each other, forming an antigen-binding site that binds to 4-1BB.

40. The formulation of claim 1, wherein the anti-PD-L1/anti-4-1BB bispecific antibody or antigen-binding fragment thereof comprises:
a first heavy chain constant region (CH1) comprising an amino acid sequence that is at least 90% identical to SEQ ID NO:8, and a first light chain constant region (CL1) comprising an amino acid sequence that is at least 90% identical to SEQ ID NO:4, wherein the CH1 and CL1 can interact with each other, forming an antigen-binding site that binds to PD-L1, and
a second heavy chain variable region (CH2) comprising an amino acid sequence that is at least 90% identical to SEQ ID NO:14, and a second light chain variable region (CL2) comprising an amino acid sequence that is at least 90% identical to SEQ ID NO:4, wherein the CH2 and CL2 can interact with each other, forming an antigen-binding site that binds to 4-1BB.

41. The formulation of claim 1, wherein the formulation has long-term stability.

42. A method of treating a subject having cancer, the method comprising administering a therapeutically effective amount of the formulation of claim 1 to the subject:
preferably, the subject has leukemia, lymphoma, myeloma, brain tumor, head and neck squamous cell cancer, non-small cell lung cancer, nasopharyngeal cancer, esophageal cancer, gastric cancer pancreatic cancer gallbladder cancer, liver cancer, colorectal cancer, breast cancer, ovarian cancer, cervical cancer, endometrial cancer, uterine sarcoma, prostate cancer, bladder cancer, renal cell cancer, melanoma, small cell lung cancer or bone cancer;
preferably, the subject is a human.

43.-44. (canceled)

45. The formulation of claim 1, wherein the formulation comprises, consists of or consists essentially of: (a) an anti-PD-L1/anti-4-1BB bispecific antibody or antigen-binding fragment thereof comprising an anti-PD-L1 arm comprising a first heavy chain variable region comprising an amino acid sequence that is at least 90% identical to SEQ ID NO:6 and a first light chain variable region comprising an amino acid sequence that is at least 90% identical to SEQ ID NO:2; and an anti-4-1BB arm comprising a second heavy chain variable region comprising an amino acid sequence that is at least 90% identical to SEQ ID NO:12 and a second light chain variable region comprising an amino acid sequence that is at least 90% identical to SEQ ID NO:10;
(b) 10 mM or 15 mM acetic acid/sodium acetate;
(c) 100 mM or 140 mM arginine (e.g., arginine-HCl);
(d) optionally 130 mM trehalose; and
(e) 0.2 mg/ml polysorbate 80,
wherein the formulation has a pH of 5.5-6.0 (e.g., 5.6 or 5.8);
preferably, the anti-PD-L1/anti-4-1BB bispecific antibody or antigen-binding fragment thereof has a concentration of about 10 mg/ml to about 100 mg/ml;
preferably, the anti-PD-L1/anti-4-1BB bispecific antibody or antigen-binding fragment thereof has a concentration of about 20 mg/ml to about 80 mg/ml;
preferably, the anti-PD-L1/anti-4-1BB bispecific antibody or antigen-binding fragment thereof has a concentration of about 20 mg/ml, about 50 mg/ml. or about 80 mg/ml.

46.-48. (canceled)


SEQUENCE LISTING (30 sequences)

SEQ ID NO: 1 - DNA, 321 bp, synthetic construct
  misc_feature 1..321: note = light chain variable region
  source 1..321: mol_type = other DNA
gacatccagatgacccagtccccttcctccctgtccgcctctgtgggcgacagggtgaccatcacctgcaaggcctcccaggatgtgcacaccgccgtggcttggattcagcagaagcccggcaagtcccccaagctgctgatctactccgcctccaaccggtacaccggcgtgcctagcaggttcagcggaagcggcagcggcaccgacttcaccttcaccatctcctccctgcagcctgaggacatcgccacctattactgccagcagcactacatcacccccctgaccttcggccagggcaccaagctggagatcaag

SEQ ID NO: 2 - protein, 107 aa, synthetic construct
  REGION 1..107: note = light chain variable region
DIQMTQSPSSLSASVGDRVTITCKASQDVHTAVAWIQQKPGKSPKLLIYSASNRYTGVPSRFSGSGSGTDFTFTISSLQPEDIATYYCQQHYITPLTFGQGTKLEIK

SEQ ID NO: 3 - DNA, 321 bp, synthetic construct
  misc_feature 1..321: note = light chain constant region
  source 1..321: mol_type = other DNA
cgtacggtggccgcccccagcgtcttcatttttccaccctctgacgaacagctgaagtcagggacagcttccgtggtctgtctgctgaacaatttttaccccagggaggccaaagtgcagtggaaggtcgataacgctctgcagagcggaaattctcaggagagtgtgacagaacaggactcaaaagattccacttatagcctgtctagtaccctgacactgtccaaggcagactacgaaaagcataaagtgtatgcctgtgaggtcacacatcagggtctgtcaagccccgtcactaagtccttcaaccggggagaatgc

SEQ ID NO: 4 - protein, 107 aa, synthetic construct [sequence omitted]

SEQ ID NO: 5 - DNA, 351 bp, synthetic construct
  misc_feature 1..351: note = heavy chain variable region
  source 1..351: mol_type = other DNA
gaagtgcagctggtgcagtccggcgccgaggtgaagaagcctggctcctccgtgaaggtgtcctgcaaggcctccggcttcaacatcgaggacacctacatccactgggtgaggcaggctcccggacagggactggagtggatgggcaggatcgatcccgccaacgccaacaccaagtacgaccccaagttccagggcagggtgaccatcaccgccgataccagcaccaacaccgcctacatggagctgtcctccctgaggtccgaggacacagccgtgtactactgtggcaggggactgggcgcctggtttgcctactggggacagggcaccctggtgaccgtgtcctcc

SEQ ID NO: 6 - protein, 117 aa, synthetic construct
  REGION 1..117: note = heavy chain variable region
EVQLVQSGAEVKKPGSSVKVSCKASGFNIEDTYIHWVRQAPGQGLEWMGRIDPANANTKYDPKFQGRVTITADTSTNTAYMELSSLRSEDTAVYYCGRGLGAWFAYWGQGTLVTVSS

SEQ ID NO: 7 - DNA, 990 bp, synthetic construct
  misc_feature 1..990: note = heavy chain constant region
  source 1..990: mol_type = other DNA
gctagcacaaaaggaccttccgtgtttccactggcaccctctagtaagagtacttcaggaggaaccgcagcactgggatgtctggtgaaggactacttcccagagcccgtcaccgtgtcttggaacagtggagcactgacctccggggtccatacatttcctgccgtgctgcagtcatccggtctgtatagcctgagctctgtggtcacagtcccaagttcatccctgggcacccagacatacatctgcaacgtgaatcacaaaccttccaatactaaggtcgacaagaaagtggaacccaagtcctgcgataagacccacacatgccctccctgtcctgctcccgaactgctgggaggaccctccgtcttcctgttcccccccaagcccaaagacacactgatgatcagcaggacccctgaagtgacctgcgtggtcgtggacgtgagccacgaggaccccgaggtcaagtttaactggtacgtggacggcgtggaggtccacaacgccaagaccaagcccagggaggagcagtacaacagcacctacagggtcgtgtccgtgctgaccgtgctccaccaagattggctcaacggcaaggagtataagtgcaaagtcagcaacaaggccctccccgcccccatcgagaaaaccatcagcaaggccaagggccaaccgcgggaacctcaagtgtataccctccctcccagccgggatgagctgaccaagaaccaagtctccctcttgtgcctggtcaagggattctacccttccgacattgccgtcgaatgggagagcaatggccagcccgagaacaactacaagacaaccccccccgtcctgcgcagcgacggatccttcttcctgtactccaagctcaccgtggacaagagccggtggcaacagggcaacgtgttctcctgtagcgtgatgcacgaagccctccacaaccactatacccagaagagcctgagcctcagccccggcaaa

SEQ ID NO: 8 - protein, 330 aa, synthetic construct
  REGION 1..330: note = heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLRSDGSFFLYSKLTVDKSRWQQGNVFSCSVMHEALHNHYTQKSLSLSPGK

SEQ ID NO: 9 - DNA, 321 bp, synthetic construct
  misc_feature 1..321: note = light chain variable region
  source 1..321: mol_type = other DNA
gacatccagatgacccagagccctagcagcctgagcgccagcgtgggcgacagagtgaccatcacctgcagagccagccaggacatcagcaattacctgaattggtaccagcagaagcctggcaaggtgcctaagctgctgatctaccacaccagcagactgcacagcggcgtgcctagcagattcagcggcagcggcagcggcaccgacttcaccctgaccatcagcagcctgcagcctgaggacgtggccacctactactgccagcagggccacaccctgcctagaaccttcggcggcggcaccaaggtggagatcaag

SEQ ID NO: 10 - protein, 107 aa, synthetic construct
  REGION 1..107: note = light chain variable region
DIQMTQSPSSLSASVGDRVTITCRASQDISNYLNWYQQKPGKVPKLLIYHTSRLHSGVPSRFSGSGSGTDFTLTISSLQPEDVATYYCQQGHTLPRTFGGGTKVEIK

SEQ ID NO: 11 - DNA, 357 bp, synthetic construct
  misc_feature 1..357: note = heavy chain variable region
  source 1..357: mol_type = other DNA
caggtgcagctggtgcagagcggcgccgaggtgaagaagcctggcagcagcgtgaaggtgagctgcaaggccagcggctacaccttcaccagctactggatcaattgggtgagacaggctccgggccaagggctggagtggatgggcaatatctaccctagcgacagctacaccaattacaatcagaagttcaaggacagagtgaccatcaccgccgacaagagcaccagcaccgcctacatggagctgagcagcctgagaagcgaggacaccgccgtgtactactgcgccagactgtactacggcagcagccctttcgactactggggacaaggcaccaccgtgaccgtgagcagc

SEQ ID NO: 12 - protein, 119 aa, synthetic construct
  REGION 1..119: note = heavy chain variable region
QVQLVQSGAEVKKPGSSVKVSCKASGYTFTSYWINWVRQAPGQGLEWMGNIYPSDSYTNYNQKFKDRVTITADKSTSTAYMELSSLRSEDTAVYYCARLYYGSSPFDYWGQGTTVTVSS

SEQ ID NO: 13 - DNA, 990 bp, synthetic construct
  misc_feature 1..990: note = heavy chain constant region
  source 1..990: mol_type = other DNA
gctagcacaaaaggaccttccgtgtttccactggcaccctctagtaagagtacttcaggaggaaccgcagcactgggatgtctggtgaaggactacttcccagagcccgtcaccgtgtcttggaacagtggagcactgacctccggggtccatacatttcctgccgtgctgcagtcatccggtctgtatagcctgagctctgtggtcacagtcccaagttcatccctgggcacccagacatacatctgcaacgtgaatcacaaaccttccaatactaaggtcgacaagaaagtggaacccaagtcctgcgataagacccacacatgccctccctgtcctgctcccgaactgctgggaggaccctccgtcttcctgttcccccccaagcccaaagacacactgatgatcagcaggacccctgaagtgacctgcgtggtcgtggacgtgagccacgaggaccccgaggtcaagtttaactggtacgtggacggcgtggaggtccacaacgccaagaccaagcccagggaggagcagtacgccagcacctacagggtggtcagcgtgctgaccgtgctgcaccaggattggctcaacggcaaggagtacaagtgcaaagtctccaacaaggccctgcccgcccccatcgagaagaccatctccaaggctaagggacagcccagggagccccaagtgtacaccgagcctcccagccgggatgagctgaccaagaaccaagtctccctcacctgcctggtcaagggattctacccttccgacattgccgtcgaatgggagagcaatggccagcccgagaacaactacaagacaaccccccccgtcctggatagcgacggatccttcttcctgctctccgtgctcaccgtcgacaagagcagatggcagcagggcaacgtgttcagctgtagcgtgatgcacgaggccctgcacaaccactacacccagaagagcctgtccctcagccccggcaag

SEQ ID NO: 14 - protein, 330 aa, synthetic construct
  REGION 1..330: note = heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTEPPSRDELTKNQVSLTCLVKGFYPSDIAVEWESNGQPENNYKTTPPVLDSDGSFFLLSVLTVDKSRWQQGNVFSCSVMHEALHNHYTQKSLSLSPGK

SEQ ID NO: 15 - protein, 10 aa, synthetic construct
  REGION 1..10: note = PD-L1 HCDR1
GFNIEDTYIH

SEQ ID NO: 16 - protein, 17 aa, synthetic construct
  REGION 1..17: note = PD-L1 HCDR2
RIDPANANTKYDPKFQG

SEQ ID NO: 17 - protein, 8 aa, synthetic construct
  REGION 1..8: note = PD-L1 HCDR3
GLGAWFAY

SEQ ID NO: 18 - protein, 11 aa, synthetic construct
  REGION 1..11: note = PD-L1 LCDR1
KASQDVHTAVA

SEQ ID NO: 19 - protein, 7 aa, synthetic construct
  REGION 1..7: note = PD-L1 LCDR2
SASNRYT

SEQ ID NO: 20 - protein, 9 aa, synthetic construct
  REGION 1..9: note = PD-L1 LCDR3
QQHYITPLT

SEQ ID NO: 21 - protein, 10 aa, synthetic construct
  REGION 1..10: note = 4-1BB HCDR1
GYTFTSYWIN

SEQ ID NO: 22 - protein, 17 aa, synthetic construct
  REGION 1..17: note = 4-1BB HCDR2
NIYPSDSYTNYNQKFKD

SEQ ID NO: 23 - protein, 10 aa, synthetic construct
  REGION 1..10: note = 4-1BB HCDR3
LYYGSSPFDY

SEQ ID NO: 24 - protein, 11 aa, synthetic construct
  REGION 1..11: note = 4-1BB LCDR1
RASQDISNYLN

SEQ ID NO: 25 - protein, 7 aa, synthetic construct
  REGION 1..7: note = 4-1BB LCDR2
HTSRLHS

SEQ ID NO: 26 - protein, 9 aa, synthetic construct
  REGION 1..9: note = 4-1BB LCDR3
QQGHTLPRT

SEQ ID NO: 27 - protein, 447 aa, synthetic construct
EVQLVQSGAEVKKPGSSVKVSCKASGFNIEDTYIHWVRQAPGQGLEWMGRIDPANANTKYDPKFQGRVTITADTSTNTAYMELSSLRSEDTAVYYCGRGLGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLRSDGSFFLYSKLTVDKSRWQQGNVFSCSVMHEALHNHYTQKSLSLSPGK

SEQ ID NO: 28 - protein, 214 aa, synthetic construct
DIQMTQSPSSLSASVGDRVTITCKASQDVHTAVAWIQQKPGKSPKLLIYSASNRYTGVPSRFSGSGSGTDFTFTISSLQPEDIATYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 449 aa, synthetic construct
QVQLVQSGAEVKKPGSSVKVSCKASGYTFTSYWINWVRQAPGQGLEWMGNIYPSDSYTNYNQKFKDRVTITADKSTSTAYMELSSLRSEDTAVYYCARLYYGSSPFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTEPPSRDELTKNQVSLTCLVKGFYPSDIAVEWESNGQPENNYKTTPPVLDSDGSFFLLSVLTVDKSRWQQGNVFSCSVMHEALHNHYTQKSLSLSPGK

SEQ ID NO: 30 - protein, 214 aa, synthetic construct
DIQMTQSPSSLSASVGDRVTITCRASQDISNYLNWYQQKPGKVPKLLIYHTSRLHSGVPSRFSGSGSGTDFTLTISSLQPEDVATYYCQQGHTLPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC